(19) United States
(12) Patent Application Publication
(10) Pub. No.: US 2005/0044592 A1
(43) Pub. Date: Feb. 24, 2005

(54) Plant growth modulation

(76) Inventors: Hilde Nelissen, Tongeren (BE); Lieven Deveylder, Drongen (BE); Jonathan Clarke, Norfolk (GB); Dirk Inze, Moorsel-Aalst (BE); Maria van Lijsebettens, Merelbeke (BE)

Correspondence Address: TRASK BRITT, P.O. BOX 2550, SALT LAKE CITY, UT, 84110, US

(21) Appl. No.: 10/915,029

(22) Filed: Aug. 9, 2004

Related U.S. Application Data
(63) Continuation of application No. PCT/EP03/01287, filed on Feb. 7, 2003.

(30) Foreign Application Priority Data
Feb. 7, 2002 (EP) 02075511.2

(51) Int. Cl. A01H 1/00; C12N 15/82

(52) U.S. Cl. 800/287; 435/468

(57) ABSTRACT

The present invention relates to the use of one or more genes, encoding a protein of the elongator complex to modulate plant growth. More specifically, the invention relates to the overexpression of the DRL1 gene, to simulate growth of leaves and roots.

### PRIORITY CLAIM

This application is a continuation of International Application Serial No. PCT/EP03/01287, filed Feb. 7, 2003, published, in English, on Aug. 14, 2003 as Publication No. WO 03/066852 A2, the contents of the entirety of which are incorporated herein by this reference.

### TECHNICAL FIELD

The present invention relates generally to biotechnology, and more specifically to the use of one or more genes, encoding a protein of the elongator complex to modulate plant growth. More specifically, the invention relates to the overexpression of the DRL-1 gene, to simulate growth of leaves and roots.

### BACKGROUND

Plants develop mainly post-germination from an embryo with a rudimentary body plan. The embryonic apical-basal axis is delineated by apical meristems that determine the future growth direction of the organism. The embryonic radial axis determines the identity and arrangement of tissues in concentric layers. During development pattern formation, growth and differentiation are overlapping rather than consecutive events. These processes are reiterated throughout the life cycle upon formation of every new organ. Axis formation is the basis for pattern formation within the whole plant body, an organ or even a tissue.

In Arabidopsis, leaves initiate post-germination at specific positions at the periphery of the shoot apical meristem according to a radial pattern imposed by the plant hormone auxin (Reinhardt et al., 2000). The repression of the homeobox gene SHOOT MERISTEMLESS and the activation of the myb gene ASYMMETRIC (AS) are crucial for leaf initiation (Long et al., 1996; Byrne et al., 2000). AS imposes a dorsi-ventral asymmetry upon the radial symmetry of the leaf primordium (Byrne et al., 2000). Dorsal identity in the leaf blade is promoted by the PHABULOSA and PHAVOLUTA transcription factors (TF) (McConnell et al., 2001) and ventral identity by the YABBY and KANADI TFs (Siegfried et al., 1999; Sawa et al., 1999; Kerstetter et al., 2001). Four tissues are specified along the dorsi-ventral axis: the upper epidermis and palissade parenchyma with dorsal identity, the spongy parenchyma and the lower epidermis with ventral identity.

In the primary root the radial axis of the radicle (embryonic root) is reinforced by positional information that originates from the top to the bottom, i.e. from mature cells to initial cells (van den Berg et al., 1995) and polar auxin transport (Sabatini et al., 1999). Tissues are arranged in concentric layers: the epidermis, the cortex, the endodermis, the pericycle and the vascular bundle. SCARECROW and SHORT ROOT are important genes for cortex specification (Scheres et al., 1995; Di Laurenzio et al., 1996), TORNADO 1 & 2 are important for epidermis specification (Cnops et al., 2000). Pattern formation in the primary root epidermal cell layer results in root hair cell files alternating with non-hair cell files which are formed at the anticlinal wall of two underlying cortex cells (Dolan et al., 1993, 1994). The gaseous hormone ethylene and auxin positively regulate root hair cell identity (Masucci et al., 1996). TRANSPARENT TESTA GLABRA1 and CAPRICE are positive regulators of root hair cell identity, GLABRA2 is a negative regulator (Di-Cristina et al., 1996; Wada et al., 1997; Walker et al., 1999).

The shoot apical meristem is essential for the formation of the vegetative plant body. Regulated cell division activity and changes in the orientation of cell plates precede the initiation of leaf primordia. Growth of leaf primordia occurs mainly along the length (proximo-distal axis) and width (centro-lateral axis) direction and is restricted along the thickness (dorsi-ventral axis) direction because of pattern formation in tissue layers. Early growth processes in leaves occur mainly by anticlinal cell divisions leading to the sheet-like structure of the blade. These growth processes are coupled with dorsi-ventral pattern formation (Siegried et al., 1999; McConnell et al., 2001; Eshed et al., 2001). Late growth occurs by cell expansion processes (Tsuge et al., 1996; Kim et al., 1998). Pattern formation in lateral growth results in the distinction between lamina and petiole (van der Graaff et al., 2000). Restriction of growth determines the final shape and size of the leaf organ. At flower induction, the SAM changes identity to an inflorescence meristem of which the structure and activity resembles that of the SAM except it produces floral meristems as lateral organs instead of leaf primordia. The onset of cell division in plants and animals is controlled at the G1/S transition of the cell cycle by the retinoblastoma protein that in a hypo-phosphorylated state binds and inactivates the general transcription factors E2F. Upon a mitogenic signal, such sucrose or cytokinin activated cyclin D/CDK complexes hyper-phosphorylate retinoblastoma and derepress E2F. By preventing cell cycle entry into S-phase, retinoblastoma plays a role in cell differentiation as well (de Jager and Murray, 1999). The cross-talk between cell cycle progression and developmental programs is a new and exciting area of research and the first reports have been published (Gaudin et al., 2000; De Veylder et al., 2001).

Regulation of gene expression at the transcriptional level is an important and universal mechanism of controlling developmental programs. Classes of specific TFs recognize upstream promoter boxes in specific sets of genes. Through direct or indirect interaction with the general TFs the RNA polymerase II (RNAPII) transcription initiation complex is either activated or repressed. The specific TFs are activated by environmental or developmental stimuli that are transduced from the cell plasma membrane into the nucleus. Evidence in yeast and humans is accumulating that the control of expression of sets of genes is also mediated by the process of transcription elongation. The RNAPII transcription elongation complex forms the unfolded structure of transcribing nucleosomes (Walia et al., 1998). The elongation reaction is stimulated by a large variety of factors of which some prevent pausing or stalling of the RNAPII complex and others model the chromatin for transcription. The degree of chromatin condensation is modulated by histone acetyltransferases and deacetylases (Walia et al., 1998; Wittschieben et al., 1999). Elongating RNAPII holoenzyme co-purified with a multisubunit complex, Elongator, whose stable interaction is dependent on the hyperphosphorylated state of the RNAPII carboxy-terminal domain (Otero et al., 1999). The elongator complex consists of two subcomplexes: one consists of ELP1 (Otero et al., 1999), ELP2, a WD40 repeat protein (Fellows et al., 2000) and ELP3, a histone acetyltransferase (Wittschieben et al., 1999), the other one of ELP4, ELP5, and ELP6 (Krogan and Greenblatt, 2001; Winkler et al., 2001). Most components of Elongator are well conserved from yeast to man (Hawkes et al., 2001). Phenotypes of elpA mutants in yeast were slow growth adaptation, slow gene activation and temperature sensitivity and demonstrated that the ELP genes play a role in the activation of inducible genes in the adaptation to new growth conditions (Wittschieben et al., 1999; Otero et al., 1999; Fellows et al., 2000; Krogan and Greenblatt, 2001; Winkler et al., 2001). Mutations in man in one of the Elongator components cause familial dysautonomia, a well-known disorder (Hawkes et al., 2001). We identified the DEFORMED ROOT AND LEAFI (DRL1) gene, a homolog of the yeast TOT4/KTI12 gene (Butler et al., 1994; Frohloff et al., 2001). TOT genes were identified in search of mutants resistant to the Kluyveromyces lactis toxin zymocin. TOT1, TOT2, and TOT3 are isoallelic to ELP1, ELP2 and ELP3 and hence TOT equals elongator. TOT4/KT112 encodes a protein that associated with the elongator complex (Frohloffet al., 2001). The tot4 mutant displays similar phenotypes as deficient elongator mutants, in addition to slow growth, G1 cell cycle delay and hypersensitivity to Calcofluor White and caffeine. We demonstrate that in higher plants DRL1 is important for pattern formation and growth processes.

### DISCLOSURE OF INVENTION

Provided is the use of a gene, or a functional fragment thereof, encoding a protein of the elongator complex to modulate plant growth. A preferred embodiment is the use of a gene encoding a protein selected from the group consisting of SEQ ID NO:2, SEQ ID NO:4, SEQ ID NO:6, SEQ ID NO:8, SEQ ID NO:10, SEQ ID NO:12 and SEQ ID NO:14 or from a group proteins with at least 40% similarity to one of these proteins, preferably at least 50% similarity, more preferably at least 60%, even more preferably at least 70%, most preferably 80%, as measured by a protein BLAST search (expressed as “positives”; Altschul et al., 1997). A protein of the elongator complex may be functionally replaced by a homologous protein from another species. The homology between functionally similar proteins of the elongator complex, belonging to different species, as measured by protein BLAST, is indeed starting from 40%.

Another preferred embodiment is the use of a gene according to the invention encoding a protein comprising SEQ ID NO:16.

A functional fragment as used here may be the promoter region of the gene, as well as the coding sequence of the gene, as well as a part of the coding sequence, encoding a functional fragment of the protein. As a non-limiting example, a functional fragment of DRL1 (SEQ ID NO:1) is a fragment comprising SEQ ID NO:15, preferably consisting essentially of SEQ ID NO:15, preferably consisting of SEQ ID NO:15.

Modulation of plant growth as used here includes, but is not limited to plant growth stimulation, such as stimulation of leaf growth and/or root growth, alteration in cell pattern, such as increase in cell length or cell width, as well as the succession of types of cells, change in plant architecture, such as the number of leaves.

These genes may be combined with the altered expression of one or more other genes, to obtain a synergetic effect. Preferably, the other genes are also encoding proteins of the elongation complex, or the other genes encode a protein involved in transcription regulation. More preferably, the other gene is a gene encoding retinoblastoma. Even more preferably, the other genes are encoding a protein selected from the group consisting of SEQ ID NO:2, SEQ ID NO:4, SEQ ID NO:6, SEQ ID NO:8, SEQ ID NO:10, SEQ ID NO:12 and SEQ ID NO:14 or from a group proteins with at least 40% similarity to one of these proteins, preferably at least 50% similarity, more preferably at least 60%, even more preferably at least 70%, most preferably 80%, as measured by a protein BLAST search.

Another aspect of the invention is a genetically modified plant, characterized by a modified plant growth compared to the non-transformed control, comprising one or more genetically modified genes encoding a protein of the elongator complex. Preferably, the gene is encoding a protein selected from the group consisting of SEQ ID NO:2, SEQ ID NO:4, SEQ ID NO:6, SEQ ID NO:8, SEQ ID NO:10, SEQ ID NO:12 and SEQ ID NO:14 or from a group proteins with at least 40% similarity to one of these proteins, preferably at least 50% similarity, more preferably at least 60%, even more preferably at least 70%, most preferably 80%, as measured by a BLAST search. Even more preferably, the genetically modified plant is overexpressing DRL1, or a protein with at least 40% similarity to DRL1, preferably at least 50% similarity, more preferably at least 60%, even more preferably at least 70%, most preferably 80% similarity to DRL1, as measured by a protein BLAST search.

Definitions

“Gene,” as used herein, refers both to the promoter region of the gene as well as the coding sequence. It refers both to the genomic sequence (including possible introns) as well as to the cDNA derived from the spliced messenger operably linked to a promoter sequence.

“Expression of a gene,” as used herein, refers to the transcription of the gene into messenger RNA.

“Overexpression of a gene” means that more messenger RNA is produced in the genetically modified plant than in an untransformed control plant, grown under the same conditions.

“Altered expression of a gene” means that in the genetically modified plant an amount of messenger RNA is produced that is significantly different from an untransformed control plant, grown under the same conditions.

“Functional fragment of a gene” refers to a fragment of a gene that can be used in a functional way. Typical functional fragments are the promoter region and the coding sequence. However, the term refers also to parts of the coding sequence that encode for a functional fragment of the protein, i.e. a domain of the protein that is functional on its own.

“Functional fragment of the protein,” as used herein, refers to a fragment of the protein that, on its own or as part of a fusion protein still retains the possibility to modulate plant growth. Typical functional fragments are fragments essential for the protein-protein interaction in the elongator complex. Specifically for DRL1, functional fragments are the conserved domains from AA 56 to 94, from AA 138 to 159 (including a GTPase G4 consensus motif) and from AA245 to 265, the ATP/GTP binding domain from AA 8 to 15, and the Calmodulin-binding domain, comprising AA 258 to 272, preferentially comprising AA 249 to 276, more preferentially comprising the C-terminal 100 AA. A preferred embodiment is a functional fragment comprising SEQ ID NO:16, preferably consisting essentially of SEQ ID NO:16, even more preferably consisting of SEQ ID NO:16.

“Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

“Promoter of a gene” as used herein, refers to a functional DNA sequence unit that, when operably linked to a coding sequence and possibly placed in the appropriate inducing conditions, is sufficient to promote transcription of the coding sequence.

“Operably linked” refers to a juxtaposition wherein the components so described are in a relationship permitting them to function in their intended manner. A promoter sequence “operably linked” to a coding sequence is ligated in such a way that expression of the coding sequence is achieved under conditions compatible with the promoter sequence.

“A protein of the elongator complex,” as used herein, means that the protein belongs to the multisubunit complex Elongator, as known to the person skilled in the art or to a protein associating with the complex. Preferentially, the protein has structural and/or functional homology with one of the proteins ELP1, ELP2, ELP3, ELP4, ELP5, ELP6 or TOT4/KT112 as described in Saccharomyces cerevisiae.

### BRIEF DESCRIPTION OF THE FIGURES

FIG. 1. drl1-2 leaf phenotype. Panels A and B: Fully grown rosette of wild-type and drl1-2, respectively (arrowheads indicate the transition between lamina and petiole). Panels C and D: Mean values of lamina area and lamina width of first and second expanded leaves with normal length, respectively (asterisks indicate statistically significant differences between mutant and wild-type (t-test, P≦0.05)). Panel E: Transverse section at the widest locations of expanded lamina of first leaf of wild-type (upper section) and drl1-2 (middle and lower section). Panel F: Mean values of cell area of upper epidermis and palisade in cleared expanded first and second leaves. Panels G and H: GUS activity of pFIL-GUS ventral marker in wild-type and in drl1-2, respectively. Panels I and J: GUS activity of pREV-GUS dorsal marker in wild-type and in drl1-2, respectively. (Key: i=intercellular space; mv=midvein; pc=palisade cells. Bars=50 μm for Panels G-J and 100 μm for Panel E.)

FIG. 2. drl1-2 meristematic defects. Panels A and B: Scanning electron micrograph of wild-type and drl1-2 SAM, respectively. Panel C: Longitudinal section through a 6-day-old SAM of wild-type. Panel D: Longitudinal section through a 9-day-old drl1-2 SAM. Panels E and F: Transverse section through a 12-day-old shoot apex of wild-type and drl1-2, respectively. Panel G: Longitudinal section through a 12-day-old primary root of wild-type (left) and of drl1-2 (middle), and whole-mount of a 12-day-old primary root of drl1-2 (right). Panel H: Primary root growth kinetics. Panels I and J: Inflorescence of wild-type and drl1-2, respectively. Panel K: Floral diagrams of wild-type (left) and drl1-2 (5 individuals). (Key: c=cotyledon; co=cortex; DAG=days after germination; ez=elongation zone; hy=hypocotyl; p=leaf primordium; p1 to p4=first to fourth leaf primordium; *=SAM. Bars=25 μm for Panels A and B; 50 μm for Panels C, D, E, F, G.)

FIG. 3. Alignments of the deduced amino acid sequences of AtDRL1 with Saccharomyces cerevisae (P34253), Schizosaccharomyces pombe (CAB66461), Drosophila melanogaster (046079), Mus musculus (BAB2263) and human (AAH12173).

FIG. 4. GUS activity in pDRL1-GUS transgenic plant lines using the method by De Block and Van Lijsebettens, 1998 (Panels A, B, C, D, E, F, L, M, N) and by Jefferson et al., 1987 (Panels G, H, I, J, K). Embryonic stages: late globular (Panel A), heart (Panel B) and torpedo (Panel C). Transverse sections through a 12 day-old shoot apex (Panels D and E). Longitudinal section through an inflorescence meristem (Panel F). Transverse section through an expanding leaf: leaf margin (Panel L), lamina (Panel M) and midvein (Panel N). Whole mount of carpels (Panel G), stamen (Panel H), petal (Panel J), sepal (Panel K) and primary root (Panel I).

FIG. 5. Effect of overexpression of DRL-1. The results are shown for two transformants (drl ox4 and drl ox10, compared with Ler, as described in the examples), as averages on two leaves. Panel A: leaf length; Panel B: leaf surface; Panel C: Ratio length/width.

FIG. 6. Graphs of the mean of the cell area in the upper epidermis, palisade layer and lower epidermis of fully expanded first and third leaves of the drlox4 and drlox10 lines. *=statistically significant difference with control (t-test, P<0.05).

FIG. 7. Palisade cell number in half a leaf blade at the widest width of fully expanded first and third leaves of the drlox10 line. *=statistically significant difference with control (t-test, P<0.05).

FIG. 8. Primary root length measurements, at different time intervals, of drlox4 and drlox10 lines and the Ler control. D=days after germination.

FIG. 9. Protein interactions of TOT4/KTI12 in yeast. Two-hybrid interactions are represented by arrows. Protein interactions detected by other methods indicated by dashed lines. Homologs in Arabidopsis were identified by using BLASTP; those detected by PSI-BLAST are indicated with an asterisk.

FIG. 10. Gene structure of the AtELP1 and 4 genes and position of the mutation in the corresponding elo mutants.

FIG. 11. Palisade cell number at the largest width of the fully expanded lamina of the first rosette leaf in the elo and drl1-2 mutants compared to the Ler control. *=statistical significant difference with the control (t-test, P<0.05).

FIG. 12. Graphs of the mean of the cell area in the upper epidermis and palisade layer of fully expanded first and third leaves of the elo mutant lines.

FIG. 13. Primary root length measurements at different time intervals of the elo mutants, drl1-2 and the Ler control. DAG=days after germination.

FIG. 14. Genetic interaction between DRL1 and AN. Panel A: Double-mutant (DM) analysis. Panel B: Semi-quantitative RT-PCR. Transcript levels of DRL1 and AN were compared in Ler, drl1-2, and drl1-4. Actin transcript was used as an internal control. Three independent repeats were done.

### DETAILED DESCRIPTION OF THE INVENTION

### EXAMPLES

Materials and Methods to the Examples

Plant Material and Growth Conditions

The drl1-1 and the drl1-3 allele were kindly provided by I. Bancroft (JIC, Norwich), resp. R. Simon (University, Koeln), elol, elo2, elo3 and elo4 were obtained from J. L. Micol (UMH, Alicante).

Isolation of DRL1 Genomic DNA

Total genomic DNA of the drl1-2 mutant, DSB1/30d2, was prepared according to Pruitt and Meyerowitz (1986) and digested with Hind3. Amongst the pool of fragments a 5.8 kb H3 fragment was generated containing the intact Ds and flanking plant DNA. This pool was ligated in conditions that favor the formation of monomeric circles. Primers to both Ds ends were added pointing outwards the Ds (primer 1: 5′-CGGGATTTTCCCATCCTACTTTCATCCCTG-3′ (SEQ ID NO:______) and primer 2: 5′-TTCGTTTCCGTCCCGCAAGTTAAATA-3′) (SEQ ID NO:______) and PCR reaction was done. A fragment of 1.4 kb was amplified and cloned in the pGEM-T (Promega) vector, a H3 digest confirmed the presence of a H3 site. The DNA sequence was determined of 682 bp plant DNA flanking the 3′ end of Ds and of 585 bp plant DNA flanking the 5′ end of Ds. 100% homology was found with a 250 bp genomic fragment flanking a tDs in the drl1-1 mutant, obtained after transactivation of the Ds from the DsB1 line (Bancroft et al., 1993).

A DRL1 genomic fragment containing the intron-less coding region was amplified on wild-type DNA (Ler ecotype) with primer 3 (5′-TTTTGTAGGCAGTGTGTTTA-3′) (SEQ ID NO:______) to the 5′ end of the 250 bp published DRL1 sequence (Bancroft et al., 1993) and primer 4 (5′-TCGTCGTTTTATGATTTTAT-3′) (SEQ ID NO:______) at the 3′ end of the gene and cloned in pGEM-T to create the plasmid pGEMT::DRL1. PCR reactions were done on 10 ng of total genomic DNA in 50 μl buffer (1.5 mM MgCl2, 50 mM KCl, 10 mM Tris-HCl, pH 8.3, 0.2 mM gelatin), containing 0.2 mM of each dNTP, 0.2 μM of each primer and 2.5 units of Taq polymerase (AmpliTaq®, Perkin Elmer). PCR conditions were: one cycle 5 minutes at 95° C., 1 minute at 50° C. and 1 minute at 72° C., and 34 cycles 1 minute at 95° C., 1 minute 50° C. and 1 minute 72° C.; primers were designed by the computer program OLIGO 4 primer analysis software (Rychlik, 1990).

DNA sequence was determined on an Applied Biosystems 37° A automated DNA sequencer using the 35S-dideoxy method (Sanger et al., 1977). Direct and reversed M13 primers were used to sequence both strands at least once. Intelligenetic's Suite software was used to assemble and analyze DNA sequence data.

Mapping of the DRL1 Gene

An RFLP was found between the genomes of the Ler and Col ecotypes using a Bcll restriction enzyme digest and the DRL1 genomic clone as a probe. The DRL1 gene has been mapped by using a set of 100 Recombinant Inbred lines (http://nasc.nott.ac.uk/new_ri_map.html).

In Vitro Calmodulin Binding

A GST-DRL1 C-terminal fusion was expressed in E. coli TOP 10F′ and the “crude” extract was purified on glutathion-agarose. After adjusting the protein solution to 1 mM CaCl₂, it was mixed batch-wise for 15 minutes at room temperature with CaM-Sepharose equilibrated with binding buffer consisting of 40 mM Tris-HCl pH 7.5, 50 mM NaCl, 3 mM MgCl₂, 0.2 mM CaCl₂, and 0.1 mM DTT according to Liao and Zielinski (1995). The slurry was packed into a column, the buffer drained and the column washed with 5 bed volumes of binding buffer. Bound proteins were eluted in buffer containing 40 mM Tris-HCl pH 7.5, 200 mM NaCl, 1 mM MgCl₂, 2 mM EGTA, and 0.1 mM DTT. Equal proportions of the Unbound, Wash, and Elution fractions were separated by SDS-PAGE and proteins detected by silver staining.

DRL1 Gene Expression Analysis by RT-PCR

QuickPrep® Micro mRNA Purification kit (Pharmacia) was used to isolate mRNA. The mRNA was extracted by homogenizing approximately 100 mg of tissue and was bound to Oligo (dT)-cellulose. The mRNA samples were treated with DNaseI during 1 hour to ensure the absence of genomic DNA. This is important since DRL1 is an intronless gene. 60-90 ng of mRNA from each sample was reverse transcribed by using the SuperScript Preamplification System for First Strand cDNA Synthesis (Gibco BRL). In Ler respectively drl1-2 and drl1-3 plants, cDNA synthesis was started from primer 5 (AGCCCCAAAATATGTTTGCATTA) (SEQ ID NO:______) and respectively primer 6 (TCGCGTTGATGATTTCTTGTGTC) (SEQ ID NO:______). Primer set p5-p7 (primer 7: CTAGACCGCAACCAAAACTATGC) (SEQ ID NO:______) is used to amplify wild-type cDNA, while primer set p6-p7 is used for the drl1-2 and drl1-3 mutant samples. In drl1-1 mutants primers 6 or a Ds primer 1 was used to start the cDNA synthesis. The cDNA was amplified with primer 8 (GTGGGCAACCTTGTAGTGGTAAG) (SEQ ID NO:______) and p6. To amplify the fragments, Taq polymerase and a 10×PCR buffer of Perkin Elmer were used. The PCR conditions were the same for each reaction: 3 minutes denaturation at 94° C., followed by 35 cycles of 45 seconds denaturation at 94° C., 45 seconds annealing at 55° C. and 2 minutes extension at 72° C., followed by a one time extension of 3 minutes. Not only the cDNA, but also the DNaseI treated mRNA from each sample was amplified with the same primer sets. Since DRL1 is an intronless gene, the genomic sequence of the gene could also serve as a template during the amplification of the cDNA, which could lead to false positive signals. Therefore the extracted mRNA was treated with DNaseI before cDNA synthesis. For each tissue PCR was conducted on the cDNA as well as on the DNaseI treated mRNA samples. All samples were blotted on a nitro-cellulose filter and hybridized with a DRL1 probe. Following amplification, the PCR products were run on a 0.8% agarose gels, blotted on to nylon membranes, then hybridized at 65° C. with a labeled DRL1 DNA probe, a PCR product amplified with primers p5-p7. The signals were visualized by analysis of the blots on a phosphoimager.

pDRL1-Gus Chimeric Construct and Histochemical Analysis

A promoter fragment of 1240 bp of the DRL1 gene, defined as pDRL1, was amplified from genomic Landsberg erecta DNA with the modified primer 9: ACTAGCGCCATGGGTTTTTAAAC (SEQ ID NO:______) containing a SphI restriction site and a modified primer 10: TAGTTACTTGGCATGCAGGTTATCTG (SEQ ID NO:______) containing an NcoI restriction site. The amplified sequence was cloned into the pGUS1 plasmid (kindly provided by J. Botterman, Aventis) as a SphI-NcoI fragment to create a translational GUS gene fusion. The pDRL1-GUS cassette was cloned as a PvuII fragment in the SmaI site of the pGSV4 plant transformation vector containing a kanamycine resistance marker (kindly provided by J. Botterman, Aventis) and transformed into E. coli JM109. The pGSV4::pDRL1::GUS plasmid was transferred to the Agrobacterium tumefaciens strain C58C1rif^{R} (pGV2260) (Deblaere et al., 1985) by triparental mating using the helper strain HB101 (pRK2013) according to Van Haute et al., 1983. Transgenic plants containing the pDRL1::GUS construct were obtained after root explant transformation of Ler plants using kanamycin selection (Valvekens et al., 1988). Histochemical staining using X-GLUC was used to assay DRL1 promoter activity in intact seedlings (Jefferson et al., 1987) and on thin sections of plastic embedded tissue (De Block and Van Lijsebettens, 1998).

Complementation Analysis Using dr11-2

The promoter-coding sequence of DRL1 was amplified from Ler DNA with Pfu polymerase using primers 11: AAGGAGAACCAAAGCCATTAGT (SEQ ID NO:______) and p 12: GCATTAGCGATTAATGAAGCTG (SEQ ID NO:______). The fragment (2576 bp) was cloned in the EcoRV site of a pGEM-5Zf(+) vector (Promega) and transformed into E. coli JM109. The DRL1 genomic sequence was cloned as NotI-NcoI fragment in the plasmid pAUX3133 (Goderis et al., 2002) and subsequently transferred to the pMODUL3337 plasmid containing a Basta selectable marker gene (Goderis et al., 2002) by endonuclease PI-PspI cloning. The pMODUL3337::DRL1 plasmid was transferred to the Agrobacterium tumefaciens strain C58C1rifR(pGV2260) (Deblaere et al., 1985) by triparental mating (Van Haute et al., 1983). The DRL1 gene was transformed into drl1-2 root explants (Valvekens et al., 1988) and transgenic shoots were selected on phosphinotricin 15 mg/l. The progeny of these transgenic shoots were germinated onto GM and the seedlings scored for the restoration of the wild-type phenotype. Root explants of WT T1 seedlings were tested for ppt resistance in a tissue culture assay: they were incubated for 4 days on CIM medium containing 15 mg/l ppt and then transferred for two weeks on SIM medium containing 15 mg/l ppt (Valvekens et al., 1988). Resistant root explants were covered with shoots whereas the sensitive controls did not develop callus or shoots.

Morphological and Cellular Characterization of the Leaves, Shoot Apical Meristems and Primary Roots

For the morphological and cellular analysis the expanded first two leaves, or the first and third leaves (as indicated) of drl1-2 (35 days) and Ler (28 days) were harvested. The whole-mounted leaves were fixed in 100% methanol and cleared in 90% lactic acid. Measurements of palisade and epidermal cell numbers were obtained from digitized camera-lucida drawings, made from the adaxial leaf surface using differential interference contrast optics on a Diaplan microscope (Leitz, Wetzlar, Germany). Image analyses were performed with the public domain Image program (version β-3b; Scion Corporation, Frederick, Md., USA). Statistical significance of the mean differences (P≦0.05) were analyzed by means of the t-test using the “Statistical package for the social sciences” (release 10.0.5) (SPSS Inc, Chicago, Ill., USA) on normally distributed data sets. In case of skewed distribution, the data were transformed to logarithmic values to normalize it.

Shoot apices and first expanded leaves were fixed in FAA (formaldehyde/acetic acid/ethanol), embedded in Historesin, serially sectioned (5 μm sections, Ralph glass knife, Reichert Jung 2040 Autocut) and stained with toluidine blue (0.05%). The number of palisade cells was counted in the microscope in several sections at the widest part of the lamina. A t-test on the means was performed by SPSS.

Twelve-day-old seedlings of drl1-2 and wild-type germinated in vitro on Hoagland medium and grown in vertical position were mounted (seedling+agar block) onto a slide and the primary roots were stained with a drop of propidium iodide (PI) solution (10 μg/ml). After addition of a cover slip the samples were visualized in a Zeiss LSM510 confocal microscope using the 543 nm excitation and 505-530 emission lines for PI.

Seeds were germinated in vitro on GM medium (Valvekens et al., 1988) solidified with phytagel (0.35%) in vertical position, every two days the position of the root tip was marked on the plate (Ler and elo4) or the plates were scanned (drl1-2). Root growth was measured during a period of 17 days.

Yeast Two-Hybrid

Vector and strains used were provided with the Matchmaker Two-Hybrid System vector (Clontech, Palo Alto, Calif., USA). The coding sequence of the DRL1 gene was amplified using the following primers: 5′-GTTTAAAAACCCATGGCGCTAGTTGT-3′ (SEQ ID NO:______) and 5′-ATTTGTATGATTAAAAGTAAGCTGCA-3′ (SEQ ID NO:______). The PCR fragment was cut with NcoI and SalI and was cloned into the multi-cloning site of pGBKT7 DNA-BD yeast expression vector (Clontech, Palo Alto, Calif., USA) resulting in the pGAD-DRL1 plasmid. The GAL4 activation domain cDNA fusion library of cell suspension was previously described (De Veylder et al., 1999). The screen to identify DRL1 interactors was assayed in the two-hybrid system by transforming the Saccharomyces cerevisiae HF7c (MATaura3-52 his3-200 ade2-101lys2-801 trp1-901 leu2-3 112 gal4-542 gal80-538 LYS2::GAL1UAS-GAL1TATA-HIS3 URA3::GAL417mers (3×)-CyC1TATA-LacZ) strain with pGAD-DRL1 and the cell suspension library using the lithium acetate method (Gietz et al., 1992). To estimate the number of independent cotransformants, 1/1000 of the transformation mix was plated on medium lacking leucin and tryptophan. The rest of the transformation mix was plated on medium to select for histidin prototrophy (Trp⁻, Leu⁻ and His⁻). Of the His⁺ colonies, the activation domain plasmids were isolated as described by Hoffman and Winston (1987). The pGAD10 inserts were PCR amplified using the primers 5′-AGGGATGTTTAATACCACTAC-3′ (SEQ ID NO:______) and 5′-GCACAGTTGAAGTGAACTTGC-3′ (SEQ ID NO:______) to determine the length of the inserts. Plasmid DNA was electroporated into Escherichia coli DH5α and the sequence of the inserts was determined. Extracted DNA was also used to retransform HF7c to test the specificity of the interaction.

Ethylene Responsiveness of the Drl1 Promoter

pDRL1-GUS plants germinated onto LNM (Smalle et al., 1997) for 7 days were transferred to medium containing ethylene blockers (LNM+75 μM AgNO₃ or LNM+7.5 μM AVG [Sigma]). After three days the plants were transferred to a medium containing an ethylene inducer (LNM+25 μM ACC). pDRL1-GUS plants grown on LNM were used as a positive control. As a control of the ethylene blocking and inducing effects, pACS1-GUS (Van der Straeten et al., 1992) and pDR5-GUS (Ulmasov et al., 1997) were grown in the conditions described above. Whole mount GUS-staining was done according to Jefferson et al., 1987.

Quantification of AN Transcript

To perform a semi-quantitative RT-PCR, total RNA was extracted from the shoot apices using TRIzol (Invitrogen). Total RNA (2 μg) was used as template to synthesize the cDNA by using the SuperScript™ First Strand synthesis system for RT-PCR (Invitrogen). To assess the levels of RNA in each sample, actin cDNA was amplified with primers 5′-GTGCCAATCTACGCGGGTTTC-3′ (SEQ ID NO:______) and 5′-CAATGGGACTAAAACGCAAAA-3′ (SEQ ID NO:______) and hybridized. The DRL1 gene was amplified using primer 5′-TCGCGTTGATGATTTCTTGTGTC-3′ (SEQ ID NO:______) and 5′-CTAGACCGCAACCAAAACTATGC-3′ (SEQ ID NO:______). The AN gene was amplified with primers 5′-TGAGACGGTGCCGTGGTATGG-3′ (SEQ ID NO:______) and 5′-GTTGCCTACTGGTGGATTCC-3′ (SEQ ID NO:______). The amplification of the cDNAs was terminated in the exponential phase of the PCR (18 cycles). The intensity of the hybridized fragments were measured with Image QuaNT version 4.1b (Molecular Dynamics).

Overexpression of DRL-1

The DRLox lines were constructed by introducing a p35S-DRL1histag construct into drl1-2. Homozygous lines were selected and used for the measurements. Expanded first and third rosette leaves were removed from the plants, scanned and image analysis was done with the program Scion image. The data were analyzed with the statistical program SPSS.

### Example 1

### Drl1-2 Mutant Isolation and Genetic Analysis

The drl1-2 mutant was originally named nrl1 (Clarke et al, 1996) and was identified as a leaf mutation when screening 250 F2 populations derived from a cross between the DsB1 line containing the Ds element cloned in the leader of the p35S-streptomycin phosphotransferase gene and marked by a p35S-hygromycin phosphotransferase II gene (Bancroft et al., 1992) and the AcTn25 line containing an Ac element with a p35S-Ac-transposase (Swinburne et al., 1992), both lines are of the Landsberg erecta (Ler) ecotype. drl1-2 mutant individuals were obtained in the F2 as full greens on a Streptomycin/Hygromycin-containing selective medium (number of the F2 population was DsB1-30). A mutant individual was crossed to wild-type Ler and the F2 analyzed: 702 WT and 217 drl1-2 were obtained showing that drl1-2 is a nuclear recessive mutation (χ2 (3:1)=0.87; P>0.05).

Genetic linkage analysis was done between the drl1-2 mutation and the tDs in this F2 population. No recombination was found analyzing 919 F2 and 38 F3 of the drl1-2 HmR class of which the maximum genetic distance was calculated as 6.6±3.3 cM (F2 data) or 1.3±1.2 cM (F3 data) (Koomeef and Stam, 1987). These data indicated that drl1-2 is likely to be induced by Ds insertion. Genomic DNA of 9 independent drl1-2 mutants was digested with HindIII and hybridized with an Ac probe: none of these lines did contain the parental Ds band of around 14 kb (Bancroft et al., 1993), instead they all contained a new band of 5.8 kb showing that germinal transposition of the Ds had occurred. The tDs had transposed into±1.2 kb HindIII fragment.

Three more drl1 alleles have been obtained. drl1-1 (Bancroft et al., 1993) and drl1-3 (R. Simon, unpublished results) have been isolated after independent Ds transactivation experiments starting from the DsB1 parental line (Table 1). drl1-4 corresponds to elo4, an EMS-induced leaf mutant (Berna et al., 1999).

### Example 2

### Phenotypes of DRL1 Alleles

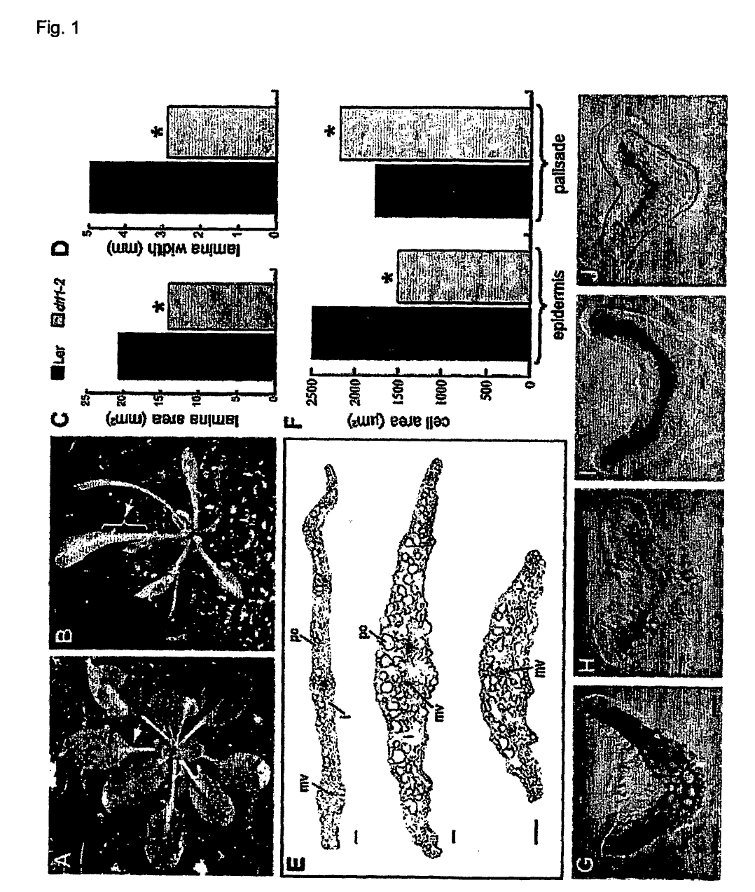

drl1-2 was isolated as a mutant with narrow leaves, compared to wild-type (FIG. 1, Panels A and B). The lamina length varied enormously among different drl1-2 individuals. The drl1-4 mutants had a less severe phenotype with significantly narrower leaf lamina, but normal leaf length, and the number (7 to 8) of rosette leaves in drl1-4 comparable to that of the wild-type, whereas it varied from 4 to 9 in drl1-2. The lamina width and area of the first and second expanded rosette leaves of a subpopulation of drl1-2 individuals with normal leaf length were measured by image analysis and they were significantly reduced when compared with the wild-type (FIG. 1, Panels C and D). The pattern formation of lateral growth along the length axis of the leaf results in a certain ratio between lamina length and petiole length. This ratio was affected in the subpopulation of drl1-2 individuals with normal leaf length, i.e. drl1-2 mutants had enlarged lamina length and reduced petiole length. In some mutant individuals no clear transition between lamina and petiole was seen (FIG. 1, Panels A and B).

In serial sections through expanded first and second leaves of drl1-2 (35-day-old seedlings), palisade cells were larger and more irregularly shaped than in wild-type and intercellular spaces were present next to the adaxial epidermis (FIG. 1, Panel E). In addition, the lateral growth was severely reduced, the lamina was thicker and the midvein less pronounced (FIG. 1, Panel E). These features may indicate ventralization of the leaf. The number of palisade cells in serial sections of an expanded leaf blade was taken as a measure for lateral growth (Tsuge et al., 1996). There were 53.4±3.4 cells in the drl1-2 mutant at the largest width (n=3), 104.2±14.1 in the drl1-4 mutant (n=4), and 112.0±5.4 cells in Ler (n=3); thus the number of palisade cells was reduced by 50% in drl1-2 and slightly reduced in drl1-4. Epidermal and palisade cells of cleared expanded first and second leaves were visualized with differential interference contrast microscopy and image analyzed. Statistically significantly smaller cells were present in the dorsal epidermis of drl1-2 (FIG. 1, Panel F) and drl1-4 mutants. The palisade layer contained significantly larger cells in drl1-2 (FIG. 1, Panel F) and drl1-4 mutants. Analysis showed that drl1-2 and drl1-4 are strong and weak alleles, respectively (Table 1). To investigate the polarity in leaves, the dorsal markers, pPHAB-gus and pREV-gus, and the ventral markers, pFIL-gus and pYAB3-gus (kindly provided by J. Bowman, University of California, Davis), were introgressed into drl1-2. These marker lines displayed promoter activity in the dorsal part of the leaf primordium, including the vascular bundles, and in the abaxial part of the leaf primordia, excluding vascular bundles, respectively (FIG. 1, Panels G and I; pPHB-gus and pYAB3-gus). Serial transverse sections of leaf primordia of F2 drl1-2 mutants containing the pREV-gus, pPHAB-gus, pFIL-gus and pYAB3-gus markers showed β-glucuronidase (GUS) activity either in the dorsal or the ventral side, similar to the parental marker lines (FIG. 1, Panels H and J; pPHB-gus and pYAB3-gus). These results demonstrate that the pattern of polarity for these genes was not altered in the drl1-2 mutant leaves, indicating that the dorsal and ventral identity was maintained. This feature was confirmed by the normal polarity in vascular bundles with adaxial xylem and abaxial phloem and the normal functional differentiation of the palisade cells, visible by the number of chloroplasts.

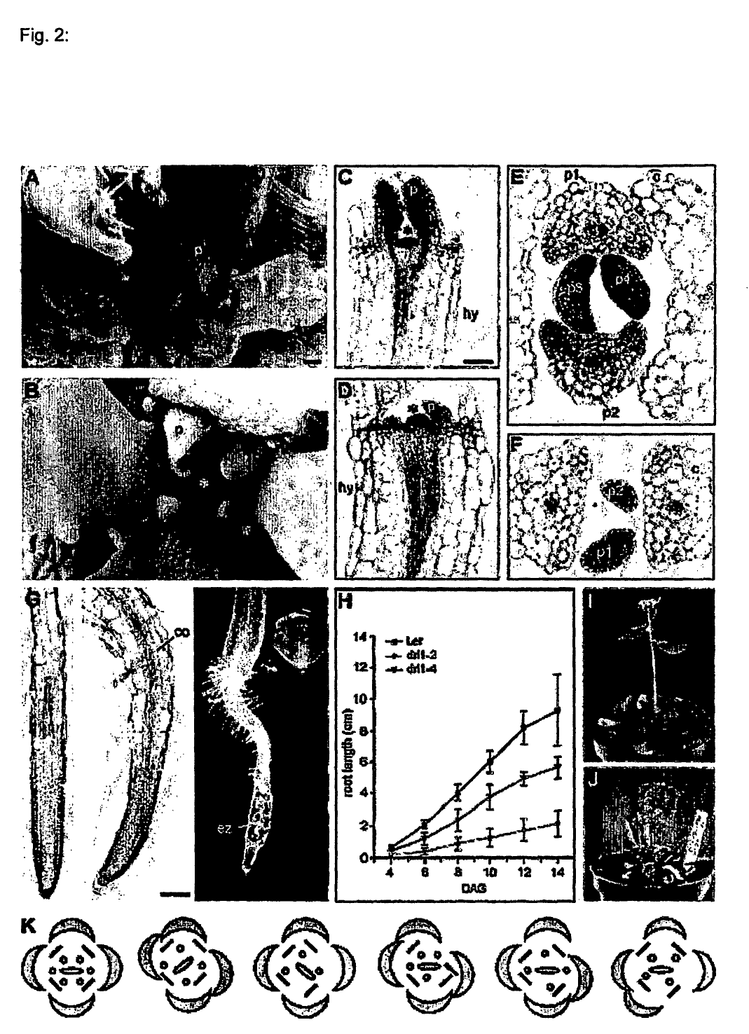

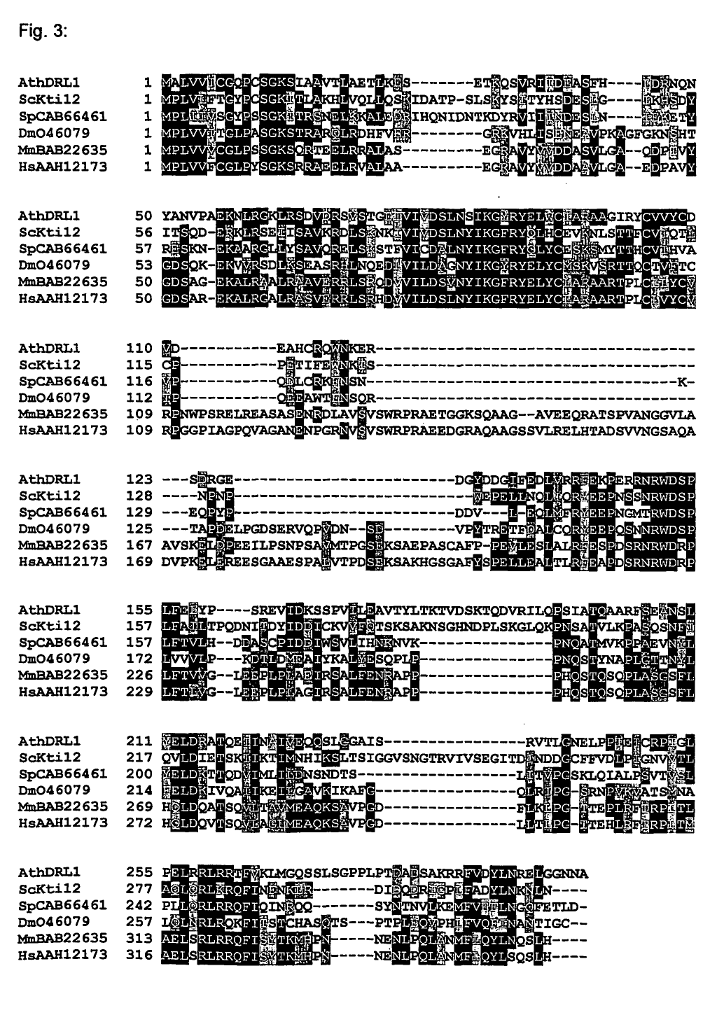

Germination of drl1-2 seeds was severely affected. Of a total of 168 seeds sown onto germination medium, 74 did not germinate (44%), 39 were seedling lethal (23%), and only 55 grew further to maturation (33%). Scanning electron microscopy and sections showed that upon emergence from the shoot apical meristem the leaf primordia were much smaller than in the wild-type (FIG. 2, Panels A, B, E, and F). This is also apparent from transverse sections through the shoot apical meristem. In addition, the mutant leaf primordia emerged more slowly than did the wild-type ones (FIG. 2, Panels C to F). Longitudinal sections through the SAM confirmed that in the mutant it was more dome-shaped than that of the wild-type (FIG. 2, Panels C and D). Transverse sections of the SAM showed that the phyllotaxis of leaves 1 and 2 of the drl1-2 was not opposite, but oblique, indicating that the pattern of leaf initiation is defective in the mutant (FIG. 2, Panels E and F). The more dome-shaped SAM, the smaller leaf primordia, and the aberrant phyllotaxis indicate that the SAM organization is defective in the drl1-2 mutant.

Primary root growth kinetics demonstrated that root growth was severely affected in drl1-2 and less defective in drl1-4 (FIG. 2, Panel H). The reduced root growth was probably related to root apical meristem defects as illustrated in longitudinal sections of 12-day-old primary roots of several drl1-2 mutant individuals (FIG. 2, Panel G). These sections also demonstrated that the cortex cells in the elongation zone were more expanded in the mutant than in the wild-type (FIG. 2, Panel G). Hypocotyl elongation was significantly reduced in the mutant, not because of a reduction in cell size (hypocotyl cells were even larger in the mutant; FIG. 2, Panels C and D), but probably because of a smaller number of cell divisions.

Flowering in drl1-2 was delayed by 1 week. Mutant inflorescences were fasciated and their size one-third that of the wild-type (FIG. 2, Panels I and J), indicating that the inflorescence meristem activity was defective. Flowers consisted of normal floral organs, but their arrangement was abnormal and the number of stamen was reduced: 4.36±0.73 in drl1-2 (n flowers=22) compared with 6 in wild-type (FIG. 2, Panel K); these defects relate to floral meristem organization.

The growth of the drl1-2 primary root is dramatically reduced and the elo4 primary root growth is intermediary between drl1-2 and Ler. The root epidermis is normally arranged in root hair forming cell files alternating with root hairless cell files. In the drl1-2 mutant root hair cell files were adjacent and hence pattern formation in the root epidermis of the mutant is defective.

Hypocotyl Elongation is Significantly Reduced in the Mutant.

### Example 3

### DRL1 Gene Isolation and Complementation

After inverse PCR on genomic DNA of the drl1-2 mutant, the sequence was determined of 682 bp plant DNA flanking the 3′ end of the tDs and of 585 bp plant DNA flanking the 5′ end of the tDs: 100% homology was found with a 250 bp genomic fragment, named DRL1, flanking a tDs in the drl1-1 mutant obtained after an independent transactivation of the Ds from the DsB1 line (Bancroft et al., 1993). The full genomic DRL1 sequence revealed one continuous open reading frame of 302 amino acids. In addition 100% homology was found between the DRL1 genomic sequence and a full-length cDNA hence the DRL1 gene is intron-less. Upon Ds insertion in drl1-2 no target site duplication had occurred in the plant DNA. The 3′ end of the tDs element is deleted by 22 bp including the terminal inverted repeat, four bases of plant DNA have been deleted and one extra C added to the 5′ side of the tDs, as a consequence no reversion events could be obtained from this allele.

The Ds insertion corresponds with AA38 in the protein sequence of drl1-1 (Bancroft et al., 1993), with AA 256 in drl1-2, and with AA 262 in drl1-3 (Table 1). In the drl1-2 mutant, the open reading frame extends 40 amino acids within the tDs. The mutation in elo4 (Berna et al., 1999) has yet to be determined. The DRL1 gene sequence was used as a probe towards mutant and wild-type plant DNAs digested with several restriction enzymes and showed that the DRL1 gene is single copy in the Arabidopsis genome.

The map position of DRL1 was determined on the RI map at the top half of chromosome 1 between the markers g12080 and 0818 (http://nasc.nott.ac.uk/new_ri_map.html). The 0818 marker corresponds to the plant DNA flanking the Ds-containing T-DNA in the DsB1 parental line. The DRL1 gene, identified after transactivation of Ds from the DsB1 line, maps at 0.06 cM distant from the 0818 marker. The Ds thus transposed over a short distance of only 12 kb, a clear example of targeting tagging. An allelism test between drl1-2 and angustifolia (Tsuge et al., 1996), a mutant with a similar leaf phenotype as drl1-2 and a map position at the top half of chromosome 1, was performed and showed that they represent two independent loci.

Partial or complete reversion events of the mutant phenotype to wild-type have been obtained from the drl1-1 allele; these were shown to be excision events of the Ds element from the DRL1 gene (Bancroft et al., 1993). We introduced the wild-type DRL1 gene with its 1240 bp promoter fragment, delineated at the 5′ end by primer 11, into the homozygous drl1-2 mutant by using a T-DNA construct containing the bar selectable marker gene conferring resistance to phosphinotricin (ppt). Seventeen independent Ti transgenic lines were obtained. They segregated wild-type to mutant seedlings in a 3 to 1 ratio or in a 15 to 1 ratio indicating one, resp. two T-DNA loci. All together 320 T2 wild-type seedlings were tested for ppt resistance they were all resistant and 33 drl1-2 seedlings were ppt sensitive showing the presence of the T-DNA containing the WT DRL1 gene and proving that complementation had occurred. These data demonstrated that the drl1-2 phenotype is due to a Ds insertion in the DRL1 gene. Our complementation analysis showed that the 1240 bp promoter fragment contained all the regulatory information to direct correct gene activity throughout the plants' life cycle.

### Example 4

### DRL1 Codes for an Homologue of the Yeast TOT4/KTI12 that Associates with Elongator

The DRL1 protein (AtDRL1) shares a high level of homology with the TOT4/KTI12 protein of baker's yeast (Saccharomyces cerevisiae) (P34253) (Butler et al., 1994; Frohloff et al., 2001). The TOT4 protein copurifies with the Elongator complex, which is important for the regulation of transcription elongation of RNAPII (Frohloff et al., 2001). Full-length genomic sequences homologous to DRL1 were obtained in Schizosaccharomyces pombe (CAB66461), Caenorhabditis elegans (Z99281), Drosophila melanogaster (046079), Mus musculus (BAB22635), Anopheles gambiae (agCP15124), human (AAH12173), Oryza sativa (cld000341.4), and Methanopyrus kandleri (NP_{—}614962). An alignment presented by Fichtner et al. (2002), showed the homology between ScP34253, SpCAB66461, CeZ99281, DmO46079, MmBAB2263, AtAAF79415, and HsAAH12173. Thus, DRL1 is not only conserved among eukaryotes, but homologs also are found in archaea, suggesting that DRL1 is a universal and ancient protein. Putative DRL1 orthologs were also identified in expressed sequence tag collections of many plant species (dicots, monocots, mosses, and conifers) and other organisms. An overview of the actual DRL1 homologs is given in Table 8.

The DRL1 protein contains a conserved ATP/GTP-binding domain (P-loop: PDOC00017 in PROSITE) ([AG]-x(4)-G-K-[ST]) spanning the amino acids 8 through 15. This domain is conserved among the homologs of TOT4 as also described by Fichtner et al. (2002). This P-loop is one of the four highly conserved sequence motifs, which are required for guanine nucleotide binding and GTP hydrolysis in GTP-binding proteins (Kaziro et al., 1991). DRL1 also contains a N[KR]XD box (amino acids 148-152), which is another conserved box of the GTP-binding protein, important for direct interaction with the guanine ring. The other two highly conserved boxes of GTP-binding proteins are not present in the DRL1 protein. A highly conserved region among DRL1 and its homologs (amino acids 194-199, PXX[AS]T) is found in many ATP or enzymes utilizing GTP (http://www.expasy.ch/tools/scanprosite/).

### Example 5

### DRL1 Binds Calmodulin in a Calcium-Dependent Manner

An in vitro assay demonstrated that the C-terminal 100 amino acids of the DRL1 protein bound calmodulin in a calcium-dependent manner. O'Neil and DeGrado (1990) showed that the binding of calmodulin to its targets is a sequence-independent recognition of amphiphilic α-helices. We found a stretch of 17 amino acids, within the C-terminal 100 amino acids of DRL1 (amino acids 257-273) that is very probably the calmodulin-binding domain. The prediction program on the calmodulin target database (Ikura, 2000) identified the same stretch as a putative CaM-binding site. Because this stretch was the only predicted CaM-binding site in the C-terminal 100 amino acids of the DRL1 protein, these amino acids comprise very probably the CaM-binding site.

Reported CaM-binding domains were compared to identify the critical elements required in the binding process. Based on the conserved hydrophobic residues within these motifs, two related motifs for calcium-dependent binding, termed 1-8-14 and 1-5-10, were described (Rhoads and Friedberg, 1997). In our proposed stretch, the motif LXXXFXXLXXXXXL and the net charge of +5 were found, according to the characteristics of an 1-8-14 CaM-binding motif of type A.

Because the CaM-binding site is sequence independent, the prediction program of the calmodulin target database (Ikura, 2000) was used to look for putative CaM-binding sites in the DRL1 homologs. No CaM-binding sites were predicted in the human, mouse, or yeast homologs. In the homolog of fruit fly, a putative CaM-binding site is also predicted at the C-terminal end of the protein. For the rice homolog, the predicted CaM-binding site also shares sequences homology with the putative CaM binding site in DRL1. These data indicate that the regulation of the DRL1 protein is conserved among plants through the binding of CaM.

### Example 6

### DRL1 Interacting Proteins are Involved in Transcription Regulation

The entire DRL1 coding sequence was used as a “bait” in a yeast two-hybrid screening using a cDNA library of cell suspensions. Of about one hundred colonies selected on Leu⁻ Trp⁻ His⁻ medium, total DNA was prepared and transformed into E. coli. The size of the inserts of the “pray” plasmids was checked by PCR and subsequently the DNA sequence was determined. Table 2 summarizes information on the type of DRL1-interacting proteins that were obtained in the yeast two-hybrid screen and were confirmed after retransformation into the DRL1-containing yeast strain and selection on Leu⁻ Trp⁻ His⁻ medium. Amongst the DRL1-interacting proteins were a histone H2A, H₂B and a histone acetyltransferase. These proteins are components of the chromatin, resp. the chromatin remodeling complexes during transcription. Their interaction with DRL1 indicates that DRL1 has a function in the transcription elongation process in analogy to its homologue, TOT4/KTI12 in yeast (Frohloffet al., 2001).

DRL1 interacts with retinoblastoma, a regulator of the E2F transcription factors that activate S-phase specific genes that promote growth by cell division (De Veylder et al., 2002), and with profilin, an interactor of E2F. DRL1 also interacts with ATH12, a member of the homeobox-leucine zipper transcription factors of which several members have been shown to control pattern formation processes during plant development. The data indicate that DRL1 is also involved in the transcription initiation processes through the interaction with transcription factors that control either growth or pattern formation in plants.

### Example 7

### The DRL1 Gene Expression is Regulated During Development

DRL1 gene expression was analyzed by RT-PCR followed by a Southern hybridization using total RNA isolated from roots, hypocotyls, cotyledons, shoot apices, stems, inflorescence apices, different developmental stages of leaves and flowers. mRNA of DRL1 was detected in every plant organ investigated in the wild-type Landsberg erecta, hence it is not organ-specific (Table 3). DRL1 is also expressed at different growth stages of Arabidopsis cell suspension cultures. In addition DRL1 mRNA was present in a mixture of leaves in different developmental stages of the drl1-2 and drl1-3 mutants, which indicates that a truncated DRL1 protein might be formed in the mutant plants. It was not possible to detect DRL1 transcript in drl1-1 mutants; this might be due to the small size of the expected PCR product (90 bp).

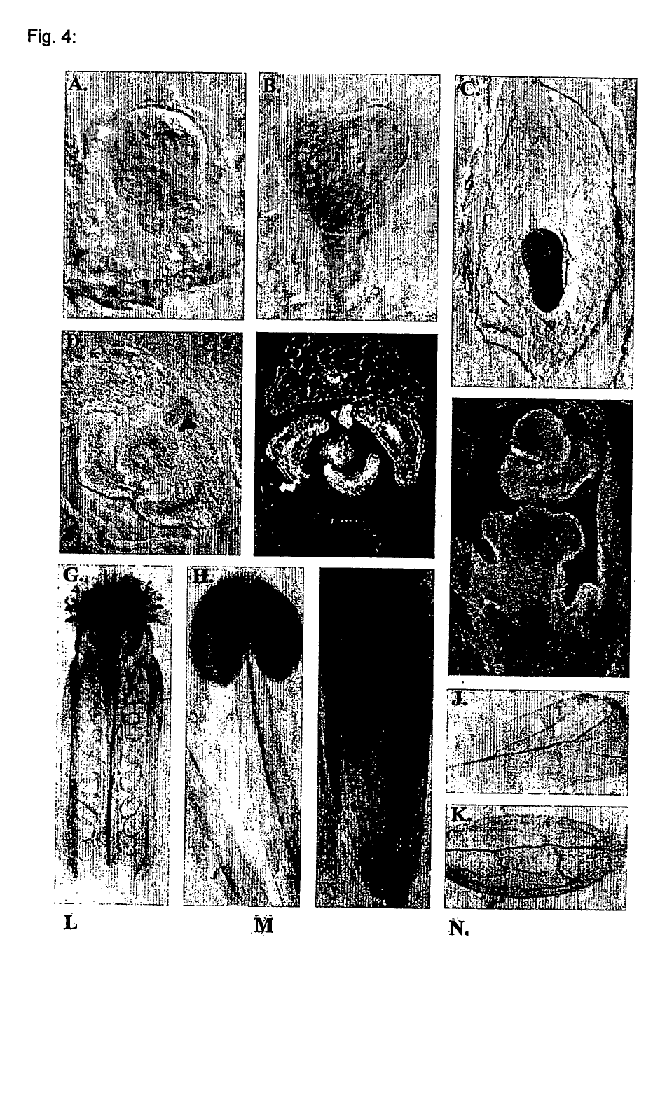

A SphI-NcoI DRL1 promoter fragment was generated after PCR amplification using primer 1 and primer 8 and it was fused at the start codon of the GUS coding sequence. This promoter fragment was used in a complementation test of the drl1-2 mutant and it was shown to contain all sequences necessary to direct complete gene activity. The DRL1 promoter activity was analyzed at the cellular level in several transgenic lines transformed with the pDRL1::GUS chimeric construct using histochemical analysis of serial transverse sections through plastic-embedded tissues (De Block et Van Lijsebettens 1998). Homogeneous GUS activity was detected in globular, heart and torpedo-stage embryos (FIG. 4, Panels A and B). High GUS activity was shown in the funiculus and the outer integument of the ovules (FIG. 4, Panel C). Other tissues were negative for X-Gluc. In transverse sections of the shoot apical meristems (n=6) of 8 to 12 day-old seedlings a ring-shaped staining was observed indicating GUS activity in the peripheral zone of the shoot apical meristem (FIG. 4, Panel D). The section more superficial of the SAM showed a circular X-Gluc staining consistent with a continuous peripheral zone in this position (FIG. 4, Panel E). The sections more distal from the SAM showed only X-Gluc staining at the periphery of the vascular bundle. Longitudinal sections through shoot and inflorescence apices showed GUS activity to be most prominent in the L2 layer, less in the L1 layer and not present in the L3 (n=3) (FIG. 4, Panel F).

In young leaf primordia the X-Gluc staining was apparent as a continuous blue linear and median area including vascular bundles and the mesophyll in between the vascular bundles (FIG. 4, Panels D and E). GUS activity occurred as a linear area conform with the basal part of the dorsal site of the leaf primordia. Transverse serial sections of expanding leaves showed a patchy GUS activity: staining was seen in individual palisade and spongy mesophyll parenchyma cells. GUS activity was absent from the mesophyll cells at the leaf tip, at the margin of the distal part of the leaf lamina and at the ventral mesophyll of the midrib (FIG. 4, Panels L, M, N). These are exactly the first parts of the leaf in which cell divisions arrest. In the leaf epidermis GUS activity was restricted to the stomatal guard cells that are generated by cell division from epidermal meristemoids after cell divisions have ceased in the epidermal pavement cells. GUS activity was also typically observed around the vascular bundles. The pDRL1::GUS pattern coincides with the patchy pattern of expression of the pcyclat::GUS during leaf development (Donnelly et al., 1999; cyc1at is equivalent to Arath;CycBl;1) and indicates that the DRL1 promoter might be cell cycle regulated.

Whole mount X-Gluc staining was done on flowers and primary roots (Jefferson et al., 1987). Young flower organs stained completely blue, fully developed sepals and petals did not show any GUS activity in analogy with fully developed leaves, a gradient of GUS activity was observed in the stamen and carpels (FIG. 4, Panels G, H, J, K, I).

### Example 8

### DRL1 Gene Expression is Responsive to Ethylene

The promoter sequence of DRL1 (the same sequence that was used in the pDRL1-GUS construct) was analyzed with the PlantCARE program (Rombauts et al., 1999) that predicts the presence of cis-acting regulatory elements, an overview is given in Table 4. The DRL1 promoter contained 15 light responsive, several cis-acting elements involved in hormone signaling (ethylene, abscisic acid, methyl jasmonate, gibberellins, salicylic acid), wound signaling and stress signaling.

The gaseous hormone ethylene is an important regulator of plant growth and development processes including germination, senescence, abscission, flowering, stress responses, cell elongation, fruit ripening and pattern formation. Ethylene regulation of DRL1 transcription was analyzed using pDRL1-GUS transgenic seedlings in a histochemical assay (Jefferson et al., 1987). The influence of exogenous ethylene was followed by using antagonists of ethylene perception (silver ions). The endogenous production of ethylene was blocked by using 1-aminoethoxyvinylglycine (AVG), an ACC synthase inhibitor. Ethylene effects were re-induced by adding a synthetic variant of ethylene, 1-aminocyclopropane-1-carboxylic acid (ACC). pDRL1-GUS plants grown on a medium containing silver ions had a severely reduced patterning of the GUS staining in the root tip, compared to transgenic plants grown on LNM, in which the root tip is highly stained. No differences were seen in plants grown on medium containing AVG. This means that the blocking of the ethylene biosynthesis had no effect on the DRL1 promoter activity, while the blocking of the exogenous ethylene perception reduced the DRL1 transcription in the root tip.

### Example 9

### Over-Repression of DRL-1

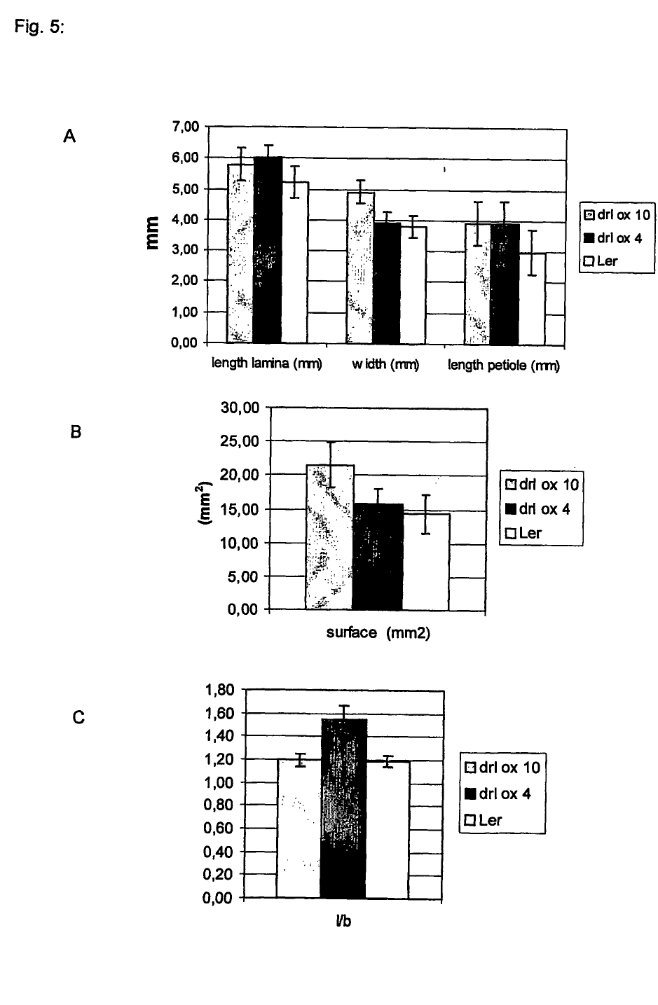

Drlox4 is a moderate DRL1-overexpressing line and Drlox10 is a highly DRL1-overexpressing line, based on Northern analysis. Both constructions have been made by overexpressing DRL1 by means of the ³⁵S promoter in the drl1-2 mutant background. The width, length and area of the leaf has been increased in the overexpressing plants, the mean values were significantly higher in the transgenic lines compared to WT (FIG. 5). Table 5 shows the significance levels of the parameters for the Drlox10 line are consistent between leaf 1 and leaf 3, i.e., they all differ significantly from the WT (except for the lamina length). The conclusion is that overexpression of DRL1 modifies the size of the leaves.

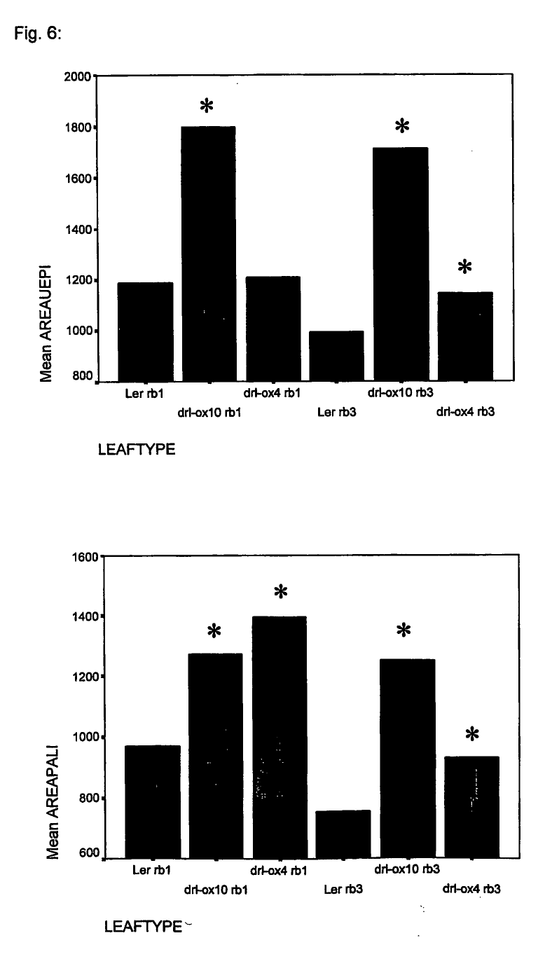

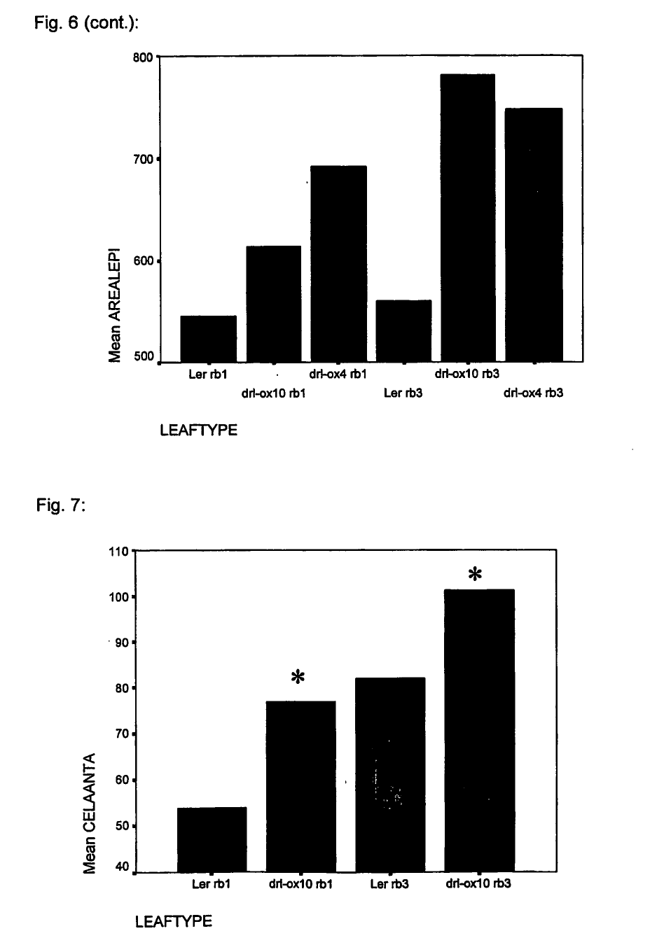

The fully-expanded leaves of the overexpression lines drlox4 and drlox10 had an increased lamina area. We determined whether this was due to an increase in cell number or cell volume or both. The cell area of the upper and lower epidermis and the palisade parenchyma was measured in the drlox lines and the results are graphically represented in FIG. 6. The cell area in the three cell layers of the third leaf were significantly larger than the Ler control and showed that an increase in cell expansion contributed to the enlarged leaf lamina area.

The palisade cell number was determined at the largest width of the lamina using serial sections. The palisade cell number was significantly increased in the drlox10 line showing that an increase in cell number contributed to the increase in lamina area (FIG. 7). The conclusion is that DRL1 plays an activating role in both growth processes of cell division and cell expansion.

Root growth kinetics was measured of the drlox lines to determine whether overexpression of the DRL1 gene induced an increased root meristem activity.

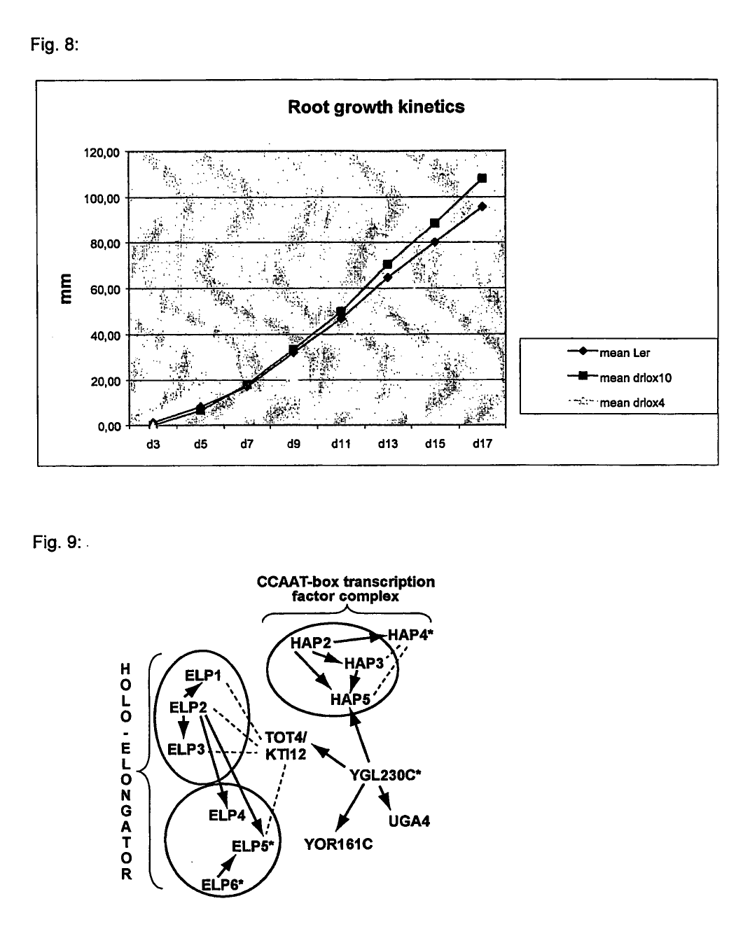

In the highest overexpression line, drlox10, there is an increased primary root growth compared to Ler (FIG. 8), however it is not statistically significant. The primary root growth in drlox4 is lower than in the Ler control indicating that the level of DRL1 overexpression in this line is not sufficient to restore wild-type root meristem activity of the drl1-2 mutation. The data indicate that at high DRL1 overexpression such as in the drlox10 line the root apical meristem activity is restored to wild-type.

The drlox10 line was back crossed (BC) to Ler wild-type and a F3 line, drlox10B5 has been selected in which the drl1-2 mutation was segregated. This BC line is being analyzed at the morphological and anatomical level to study the effect of the overexpression in a wild-type background. The palisade cell number at the widest width was counted in half a leaf blade of fully expanded first and third leaves and was significantly increased in the drlox10B5 line (leaf 1: 72.9 cells±2.6; leaf 3: 94.9 cells±6.3) compared to the Ler control (leaf 1: 55.5 cells±3.4; leaf 3: 82.0 cells±12.1). The data confirm that overexpression of the DRL gene increases cell number in leaves.

### Example 10

### Interacting Proteins of Yeast TOT4/KT112 and Their Homologs in Arabidopsis

In yeast, protein-protein interactions by two-hybrid analysis were done on a large scale to define molecular networks. Two web sites with this information (http://mips.gsf.de/proj/yeast/CYGD/db/index.html/ and http://yeast.cellzome.com/) were used to unravel the network of proteins that interact with TOT4/KTI12. An analysis of protein-protein interaction was also described by Uetz et al. (2000). TOT4/KT112 interacted with YGL230c as bait and, in addition, the latter interacted with three more proteins, UGA4, YOR161, and HAP5. UGA4 (amino acid permeability) and YOR161 (unknown function) are not discussed further. The BLASTP program of the TAIR site (http://www.arabidopsis.org/) was used to find the Arabidopsis homologs of the yeast Elongator components and the TOT4/KTI12-interacting proteins. Besides DRL1 (At1g13870), homolog of TOT4/KTI12, Arabidopsis homologs were identified: At5g13680 (ELP1), At1g49540 (ELP2), At5g50320 (ELP3), At3g11220 (ELP4, high E-value of 0.005), HAP2-3-5 homologs (Edwards et al., 1998), At1g25500 (YOR161C), and At2g01170 (UGA4) (FIG. 9). Using the PSI-BLAST program (Altschul et al., 1997), Arabidopsis homologs were found for ELP5 (At2g18410), ELP6 (At4g10090) (Ponting, 2002), YGL230C (At4g23860), and HAP4 (At5g25820) (FIG. 9, asterisks). The psi-BLAST for HAP4 was done with the Kluyveromyces lactis homolog (AF072675).

### Example 11

### Functional Analysis of A. thaliana Homologs of the Yeast Elongator Components

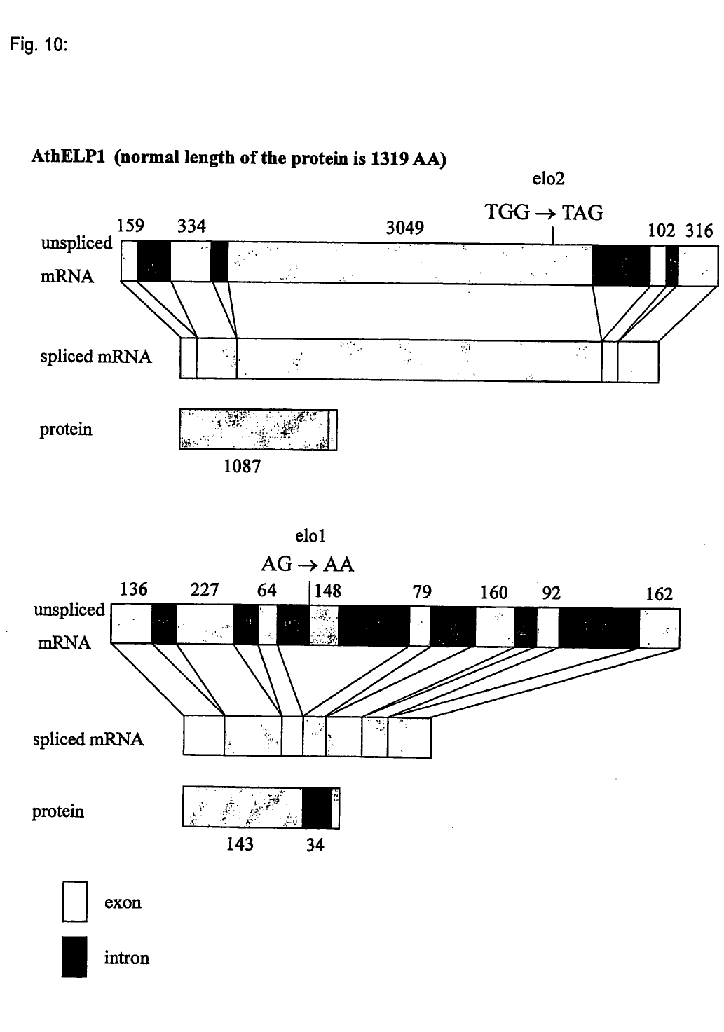

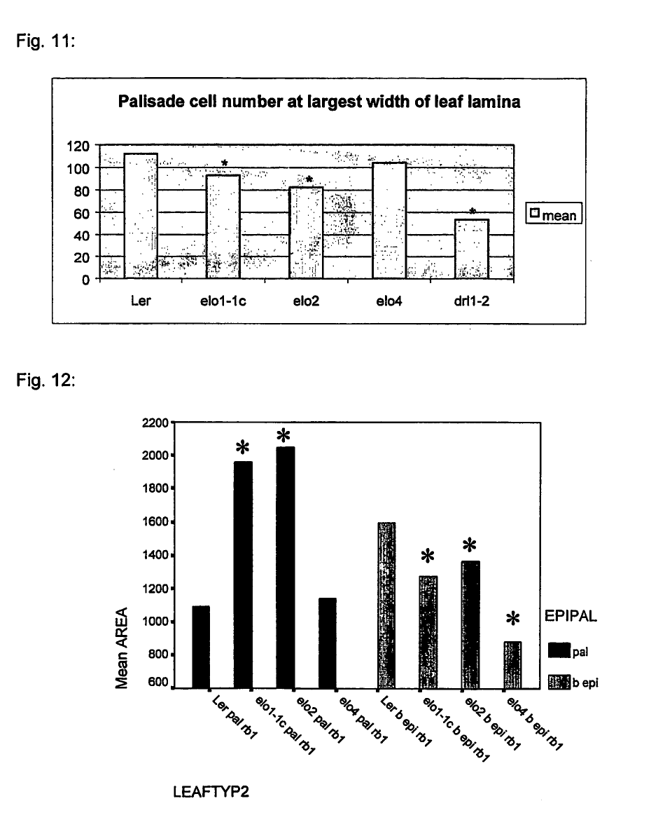

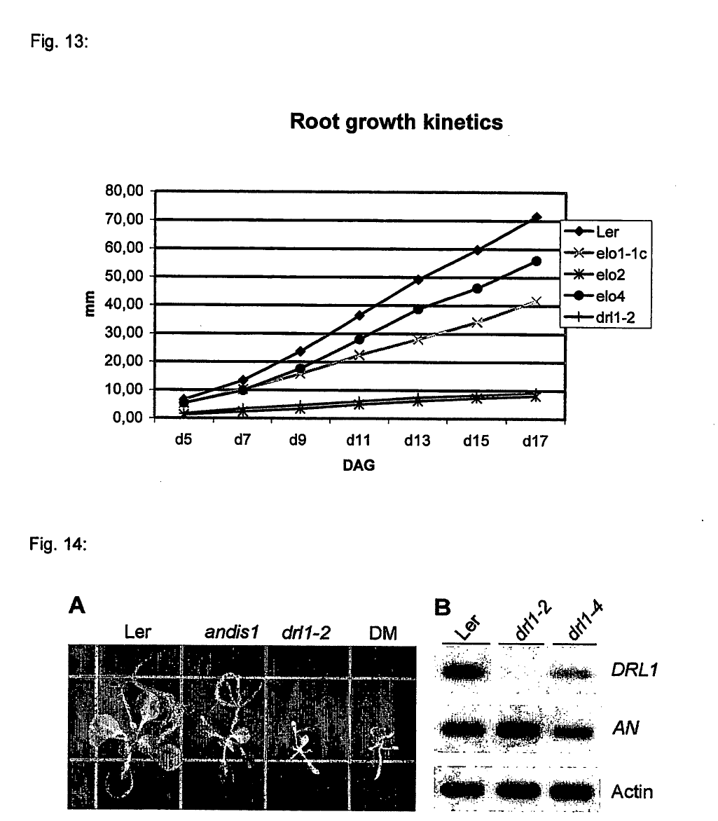

The elo class of narrow leaf mutants (Berna et al., 1999) consists of 4 independent loci that have been mapped onto the chromosomes (Robles and Micol, 2001). The map positions were compared to the genomic positions of the Arabidopsis homologues of the yeast Elongator components. The elo2 and elol mutations were located in the same region as AtELP1, resp. AtELP4. The DNA sequence of these genes was determined in the respective mutants and they contained a point mutation as indicated in Table 6. The mutation in AtELP 1 changed a TGG into the stop codon TAG resulting in a truncated protein of 1087 AA (FIG. 10). The mutation in AtELP4 changed a AG into a AA at the splice acceptor site of the third intron, resulting in the selection of the next splice acceptor site. As a consequence the fourth exon was deleted in the cDNA (which was verified experimentally) and a frame shift occurred with a precocious stop codon (FIG. 10). The elo4 mutation (Berná et al., 1999; Robles and Micol, 2001) was shown to be allelic to drl1 (Table 1). In elo4, a single base change introduced a premature stop codon at amino acid 194 (a C-to-T change at the nucleotide level at position 579). The leaves of elo1 and elo2 were analyzed by serial sectioning, image analysis and DIC optics. The leaves were narrower due to a reduction in cell number in elo1 and elo2 (FIG. 11), counting the number of palisade cells in the widest part of the lamina as a measure for cell number as described by Tsuge et al., 1996. The cell area was reduced in the epidermis and increased in the palisade cell layer in analogy to the drl1-2 mutation (FIG. 1; FIG. 12). The weak elo4 (drl1-4) allele had only a significant effect in the epidermis (FIG. 12). The root growth kinetics was measured during 17 days after germination and showed that the growth was reduced in the elo and drl1-2 mutants (FIG. 13). The leaf and root analyses showed similar phenotypes between the elo mutants and the drl1-2 mutant and provided evidence for a similar function in organ growth and the existence of a functional Elongator complex in plants. Double mutant analyses are being done to further strengthen this hypothesis.

### Example 12

### Overexpression Experiment of the Arabidopsis ELP Homologues

In yeast the Elongator complex consists of 6 proteins, ELP1-ELP6. In Arabidopsis however only for four subunits of the Elongator complex homologues can be found (ELP1-ELP4). ELP2 encodes a protein, which contains WD 40 repeats, important for protein-protein interactions. ELP3 encodes a histon acetyl transferase. The ELP2 and ELP3 Arabidopsis homologues, as well as DRL1 and the DRL1 interactors are placed under control of the constitutive 35S promoter (p35S); by means of the Gateway technique a plant transformation vector containing the p35S-gene constructs is made. Transgenic overexpression lines are constructed by introducing these constructs into plants using the floral dip method. Homozygous overexpression lines are analyzed and subsequently crossed with homozygous lines containing different overexpression constructs. The homozygous overexpression plants or the combinations of two different types of overexpressing plants show a modulated plant architecture and growth.

### Example 13

### Genetic Interaction Between DRL1 and ANGUSTIFOLIA

A double mutant analysis was done between drl1-2 and angustifolia distorted trichomes1 (an dis1), a mutant with a leaf phenotype similar to that of drl1-2, in addition to a trichome mutation, dis1, in the an background. The N2 line homozygous for the andis1 mutations in a Ler background was obtained from the Nottingham Arabidopsis Stock Center (Nottingham, UK). Compared to the drl1-2 mutants, an mutants have normal roots, flowers, and inflorescences, but wrinkled siliques. These four phenotypic differences between the two mutants were used to analyze the characteristics of the double mutant. Homozygous an F2 plants that were heterozygous for drl1-2 (selected for hygromycin resistance) were self-fertilized and analyzed in the F3 population: 1 double mutant with a drl1-2 phenotype segregated to 3 an homozygous plants. From the comparison of the vegetative phenotype of the Ler, drl1-2, an dis1, and the drl1-2—an dis1 double mutant, it could be concluded that drl1-2 is epistatic to an.

The level of AN transcript in a drl1-2 background was assessed by means of semi-quantitative RT-PCR. The AN expression levels were compared to the amount of actin transcript (FIG. 14). In the strong drl1-2 allele with severely reduced DRL1 transcript, a significant up-regulation of the AN transcript (up to 2-fold) was seen. In the weak drl1-4 allele with slightly reduced DRL1 transcript, the level of AN transcript was comparable with wild-type. The up-regulation of AN transcript in the drl1-2 allele suggests that DRL1 acts as a repressor for AN expression. Double mutant analysis showed that DRL1 acts upstream of AN, a transcriptional corepressor that regulates polar expansion of palisade cells, probably by controlling the arrangement of cortical microtubuli (Kim et al., 2002). The up-regulation in drl1-2 shoot apices of AN transcript could explain the increase in palisade cell size in the drl1-2 mutant without affecting the polarity. Indeed, recessive mutation at the AN locus results in a reduction of palisade cell size (Tsuge et al., 1996).

In yeast, Elongator was described as a histone acetyltransferase complex associated with the elongating form of RNAPII to facilitate transcription elongation (Otero et al., 1999). The RNAPII transcription elongation complex is believed to control gene expression by remodeling the chromatin through histone acetylation (Winkler et al., 2002) and by regulating the movement along the DNA (Kim et al., 2002b). Although Elongator was not detected on promoters or open reading frames in vivo (Pokholok et al., 2002), numerous data suggest a role for Elongator in transcriptional regulation. Elongator was originally found stoichiometrically associated with the elongating form of RNAPII and to bind preferentially to the hyperphosphorylated form of RNAPII in vitro (Otero et al., 1999). Mutations in transcription-elongating machinery confer increased sensitivity to the drug 6-AU, as was also seen in the tot mutants (Shaw and Reines, 2000). Moreover, microarray analysis using deletion mutants of Elongator components (ELP1, ELP2, ELP4, and ELP6) revealed that subsets of genes were down- or up-regulated, indicating that Elongator is important to regulate the expression of specific sets of genes (Krogan and Greenblatt, 2001). In Arabidopsis, we propose that Elongator regulates transcription of several genes involved in specific processes during development. In the drl1-2 mutant, the AN transcript is up-regulated suggesting an inhibitory function of DRL1 on AN transcription. Several of the drl1 phenotypes also suggest an activating or inhibitory role of DRL1 on specific processes such as meristem activity and organ growth.

### Example 14

### cDNA AFLP Transcript Profiling of the elo Mutants

cDNA-AFLP analysis was performed (Breyne et al., 2002) on total RNA prepared from shoot apices of two week-old elo1, elo2 and elo4 seedlings including the shoot apical meristem (SAM), the first and second rosette leaf in the expansion stage and the third and fourth leaf in the primordium stage. The transcriptomes of the elo mutants were compared to Ler (wild-type). Fourteen primer combinations were used to amplify approximately 1000 AFLP fragments (transcripts) and were analyzed on polyacrylamide gels. Around 10 transcripts were differentially regulated in the elo mutants compared to Ler. The same fragments were up- or down-regulated in elo1, elo2 and elo4 (Table 7), indeed a similar molecular phenotype is expected for components of one complex. The number of differentially regulated fragments was consistent with the severity of the elo phenotype: most severe phenotype in elo2, intermediate in elo1 and weak in elo4. Ectopic bands such as present in mutant and absent in wild-type or the reverse were not seen. The low frequency of 1% of transcripts with altered expression in the Elongator mutants suggests a selective regulatory function in transcription for the Elongator complex in plants.

TABLE 1
Drl alleles

| | | | Amino | | |
|---|---|---|---|---|---|
| | | | acid | Phe- | |
| | | | Position of | notypic | |
| Locus | Alleles | Mutagen | mutation | strength | Source mutation |
| DRL1 | drl1-1 | tDs | AA38 | strong | Bancroft et al., 1993 |
| | drl1-2 | tDs | AA256 | strong | This paper |
| | drl1-3 | tDs | AA262 | strong | R. Simon |
| | drl1-4 = | EMS | AA194 | weak | Berna et al., 1999 |
| | elo4 | | | | |

N.D., not determined

TABLE 2
DRL1-interacting proteins obtained in a yeast two-hybrid screening.

| | number of | | number of |
|---|---|---|---|
| general processes | clones | specific proteins | clones |
| Transcription initiation | 4 | RB-related protein | 2 |
| | | profilin | 1 |
| | | ATHB12 | 1 |
| ribosomal proteins | 14 | | 14 |
| protein modification/ | 8 | Ubiquitin | 5 |
| degradation | | protease inhibitor | 1 |
| | | phospholipase D | 1 |
| | | protein folding | 1 |
| mRNA processing | 2 | | 2 |
| Photosynthesis | 4 | electron transport | 2 |
| | | RUBISCO small | 1 |
| | | subunit | |
| | | photosystem I subunit | 1 |
| membrane traffic and | 5 | protein targeting | 2 |
| protein transport | | vesicular transport | 3 |
| Chromatin | 2 | histone H2A | 1 |
| | | Histone H2B | 1 |
| | | HAT | 1 |
| unknown/putative | 13 | | 13 |
| proteins | | | |
| signaling proteins | 5 | receptor like kinase | 1 |
| | | phosphate transporter | 1 |
| | | ATP synthase subunit | 2 |
| | | ATP/GTP binding | 1 |
| | | protein | |
| Translation | 2 | elongator factor 1 | 2 |
| C-metabolism | 5 | sucrose | 1 |
| | | glucose | 1 |
| | | other | 3 |
| cell wall | 3 | | 3 |
| electron transport | 3 | | 3 |
| cation channels | 1 | ankyrin | 1 |
| alkaloid biosynthesis | 1 | strictosidin synthase | 1 |
| cytochrome p450 | 1 | | 1 |
| spindle pole body | 1 | | 1 |
| nuclear export factor | 1 | | 1 |

TABLE 3
DRL1 gene expression measured by RT-PCR.

| | | Age (days | | |
|---|---|---|---|---|
| | | after | | DRL1 |
| | | germination/ | Developmental/ | expres- |
| Genotype | Organs/tissue | inoculation) | growth stage | sion |
| wild-type | roots | 21 | | + |
| (Ler) | | | | |
| | hypocotyls | 10 | | + |
| | cotyledons | 10 | | + |
| | shoot apices | 10 | | + |
| | young first | 10 | | + |
| | leaves | | | |
| | expanded first | 14 | | + |
| | leaves | | | |
| | mix of leaves | 21 | | + |
| | stems | 28 | | + |
| | inflorescence | 28 | | + |
| | meristems | | | |
| | flower buds | | flower stage 6-12 | + |
| | flowers | | flower stage 13-16 | + |
| | young siliques | | flower stage 17 | + |
| | full-grown | | flower stage 18 | + |
| | siliques | | | |
| drl1-1 | mix of leaves | 35 | | − |
| drl1-2 | mix of leaves | 35 | | + |
| drl1-3 | mix of leaves | 35 | | + |
| wild-type | cell suspension | 2 | early exponential | + |
| (Col) | | | | |
| | | 5 | mid exponential | + |
| | | 8 | late exponential | + |
| | | 12 | log | + |

TABLE 4
cis-regulatory elements in the DRL1 promoter
determined by the program PlantCARE.

| Function | site name | position |
|---|---|---|
| sequence conserved in alpha- | A-box | −405 |
| amylase promoters | | −187 |
| | | −96 |
| cis-acting element involved in the | ABRE | −918 |
| abscisic acid responsiveness | | −443 |
| | | −410 |
| cis-acting element involved in | ACE | −1278 |
| light responsiveness | | −443 |
| part of module for light response | AE-box | −710 |
| | | −637 |
| | | −259 |
| part of module for light response | AT1-motif | −1009 |
| part of a conserved DNA module involved | ATC-motif | −160 |
| in light responsiveness | | |
| part of a conserved DNA module involved | ATCC-motif | −162 |
| in light responsiveness | | |
| fungal elicitor responsive element | Box-W1 | −774 |
| cis-acting regulatory element involved | CGTCA-motif | −1281 |
| in the MeJa-responsiveness | | −943 |
| elicitor-responsive element | ELI-box 3 | −1264 |
| | | −5 |
| ethylene-responsive element | ERE | −1065 |
| | | −1044 |
| | | −53 |
| cis-acting regulatory element involved in | G-box | −1278 |
| light responsiveness | | −1212 |
| | | −1176 |
| | | −1174 |
| | | −583 |
| | | −442 |
| | | −441 |
| | | −427 |
| part of a light responsive element | GA-motif | −674 |
| | | −405 |
| | | −395 |
| part of a light responsive element | GATA-motif | −407 |
| | | −144 |
| cis-regulatory element involved in | GCN4_motif | −791 |
| endosperm expression | | |
| light responsive element | GT1-motif | −1153 |
| | | −1152 |
| | | −1110 |
| | | −1109 |
| | | −992 |
| cis-acting element involved in heat stress | HSE | |
| responsiveness | | |
| part of light responsive element | I-box | |
| part of light responsive element | LAMP-element | −1231 |
| | | −727 |
| | | −191 |
| MYB binding site involved in light | MRE | −1233 |
| responsiveness | | −922 |
| gibberellin-response element | P-box | −1242 |
| cis-acting element involved in | TATC-box | −906 |
| gibberellin-responsiveness | | −532 |
| cis-acting element involved in salicylic | TCA-element | −714 |
| acid responsiveness | | |
| part of a light responsive element | TCCC-motif | −1239 |
| cis-acting regulatory element involved | TGACG-motif | −1281 |
| in MeJa-responsiveness | | −943 |
| wound-responsive element | WUN-motif | |
| part of a light responsive element | chs-CMA1a | −1144 |
| | | −1094 |
| | | −480 |
| part of a light responsive element | chs-CMA2a | −1175 |

TABLE 5
T-test on morphological data of expanded leaves of
DRL1-overexpressing lines

| | | | Drlox4 | | Drlox10 | | Drlox10 | |
|---|---|---|---|---|---|---|---|---|
| | Drlox4 | | Leaf 3 | | Leaf 1 | | Leaf 3 | |
| Parameter | Leaf 1 Sig. | H₀ | Sig. | H₀ | Sig. | H₀ | Sig. | H₀ |
| Length lamina | 0.003 | NA | 0.031 | NA | 0.051 | A | 0.152 | A |
| Width lamina | 0.479 | A | 0.011 | NA | 0.000 | NA | 0.010 | NA |
| Length petiole | 0.010 | NA | 0.109 | A | 0.015 | NA | 0.016 | NA |
| Total length | 0.001 | NA | 0.038 | NA | 0.005 | NA | 0.036 | NA |
| Area lamina | 0.257 | A | 0.040 | NA | 0.001 | NA | 0.001 | NA |

A significance of >0.05 means that the null hypothesis is accepted (A) and that the parameter does not differ between WT and overexpressing line. In case P<0.05 the null hypothesis is not accepted (NA), in all cases the mean values of the transgenic lines were higher than those of WT.

TABLE 6
Arabidopsis homologs of the yeast Elongator components and
identification of their corresponding mutants.

| | | | | Length | Position | |
|---|---|---|---|---|---|---|
| S. cerevisiae | A. thaliana | | A. thal. | protein | mutation | |
| gene | homolog | Protein type | mutant | (AA) | (AA) | Phenotype |
| ELP1/ | At5g13680 | Unknown | elo2 | 1319 | 1087 | Narrow |
| TOT1 | | | | | | leaves due to |
| | | | | | | reduced cell |
| | | | | | | number. |
| | | | | | | Reduced |
| | | | | | | root growth |
| ELP2/ | At1g49540 | Hypothetical | | | | |
| TOT2 | | with (WD40) | | | | |
| ELP3/ | At5g50320 | Histon acetyl | | | | |
| TOT3 | | transferase | | | | |
| ELP4 | At3g11220 | Hypothetical | elo1 | 355 | 143 | Narrow |
| | | | | | | leaves due to |
| | | | | | | reduced cell |
| | | | | | | number. |
| | | | | | | Reduced |
| | | | | | | root growth |
| ELP5/ | At2g18410 | Hypothetical | | | | |
| TOT5 | | | | | | |
| ELP6 | At4g10090 | Hypothetical | | | | |
| TOT4/ | DRL1/ | ATP/GTP and | elo4 | 302 | 194 | Narrow |
| KTI12 | At1g13870 | CaM binding | drl1-2 | | 256 | leaves due to |
| | | | | | | reduced cell |
| | | | | | | number. |
| | | | | | | Reduced |
| | | | | | | root growth |

AA, amino acids
elo mutants (Berna et al., 1999)
drl1-2 mutants (Nelissen et al., 2003)

TABLE 7
Differentially regulated transcripts between elo mutants and the Ler
control in a cDNA AFLP analysis using 14 primer combinations.

| Transcript level | | | |
|---|---|---|---|
| compared to Ler | elo1 | elo2 | elo4 |
| higher | 9 | 10 | 6 |
| lower | 3 | 3 | 2 |
| ectopically present | 0 | 0 | 0 |
| ectopically absent | 0 | 0 | 0 |

TABLE 8
Homology of DRL1 with proteins of other phyla

| | Species | Accession | | | |
|---|---|---|---|---|---|
| Class | Similarity | number | Genomic/EST % | | Identity % |
| Eukaryota | | | | | |
| Plants dicots | Arabidopsis thaliana | AJ428870 | Genomic | 100 | 100 |
| | Medicago truncatula | AW560006 | EST | 76 | 87 |
| | Glycine max | BH021350 | EST | 68 | 82 |
| | Gossypium arboreum | BF276634 | EST | 64 | 76 |
| | Euphorbia esula | BE231335 | EST | 70 | 82 |
| | Lotus japonicus | AV420673 | EST | 74 | 88 |
| | Mesembryant. crystallinum | BF480675 | EST | 72 | 83 |
| | Lycopersicon esculentum | BI930978 | EST | 72 | 84 |
| Plants monocots | Zea Mays | AI920610 | EST | 68 | 82 |
| | Triticum aestivum | BF428902 | EST | 62 | 76 |
| | Oryza sativa | cld000341.4 | genomic | 66 | 76 |
| | Hordeum vulgare | BF616626 | EST | 62 | 77 |
| Plants conifers | Pinus taeda | BE451838 | EST | 66 | 80 |
| Plants mosses | Physcomitrella patens | AW145049 | EST | 65 | 73 |
| Birds | Gallus gallus | BG713512 | EST | 44 | 68 |
| Fish | Ictalurus punctatus | IpHdk02331 | EST | 47 | 67 |
| Molluscs | Crassostrea virginica | BG624862 | EST | 41 | 55 |
| Amphibians | Xenopus laevis | AW643264 | EST | 39 | 62 |
| Nematodes | Caenorhabditis elegans | Z99281 | genomic | 33 | 54 |
| Fungi | Schizosacch. pombe | CAB66461 | genomic | 31 | 50 |
| | Saccharomyces cerevisiae | Z28110 | genomic | 29 | 46 |
| Flies | Drosophila melanogaster | O46079 | genomic | 27 | 46 |
| | Anopheles gambiae | agCP15124 | genomic | 28 | 48 |
| Mammals | Mus musculus | BAB22635 | genomic | 26 | 43 |
| | Homo sapiens | AAH12173 | genomic | 27 | 42 |
| archaea | | | | | |
| Methanopyrus | Methanopyrus kandleri | NP_614962 | genomic | 25 | 41 |

The proteins were identified either as genomic sequences (genomic) or as partial cDNA (expressed sequence tag [EST]). In case of the partial cDNAs, identity and similarity were given for the strengths of cDNA, which encodes that part of the protein with the highest homology to DRL1.

### REFERENCES

Altschul, Stephen F., Thomas L. Madden, Alejandro A. Schäffer, Jinghui Zhang, Zheng Zhang, Webb Miller, and David J. Lipman (1997). Gapped BLAST and PSI-BLAST: a new generation of protein database search programs. Nucleic Acids Res. 25, 3389-3402.
Bancroft, I., Bhatt, A. M., Sjodin, C., Scofield, S., Jones, J. D. G., and Dean, C. (1992). Development of an efficient two-element transposon tagging system in Arabidopsis thaliana. Mol. Gen. Genet. 233, 449-461.
Bancroft, I., Jones, D. J., and Dean, C. (1993). Heterologous transposon tagging of the DRL1 locus in Arabidopsis. Plant Cell 5, 631-638.
Berna, G., Robles, P., and J. L. Micol (1999). A mutational analysis of leaf morphogenesis in Arabidopsis thaliana. Genetics 152: 729-742.
Butler, A. R., White, J. H., Folawiyo, Y., Edlin, A., Gardiner, D., and Stark, M. J. (1994). Two Saccharomyces cerevisiae genes which control sensitivity to G1 arrest induced by Kluyveromyces lactis toxin. Mol. Cell. Biol. 14, 6306-6316.
Breyne P, Dreesen R, Vandepoele K, De Veylder L, Van Breusegem F, Callewaert L, Rombauts S, Raes J, Cannoot B, Engler G, Inzé D, and Zabeau M (2002). Transcriptome analysis during cell division in plants. Proc. Natl. Acad. Sci. 99: 14825-14830.
Byrne, M. E., Barley, R., Curtis, M., Arroyo, J. M., Dunham, M., Hudson, A., and Martienssen, R. A. (2000). Asymmetric leaves 1 mediates leaf patterning and stem cell function in Arabidopsis. Nature 408, 967-971.
Clarke, J., De Bleeckere, M., Royaert, S., Vanderhaeghen, R., Van Montagu, M., and Van Lijsebettens, M. NARROW LEAF1, a gene controlling leaf lamina formation in Arabidopsis. Abstract presented at the 10th FESPP Congress on “From molecular mechanisms to the plant: an integrated approach,” Firenze (Italia), Sep. 9-13, 1996. [Plant Physiol. Biochem., Special issue, 54 (SO4-21)].
Cnops, G., Wang, X., Linstead, P., Van Montagu, M., Van Lijsebettens, M., and Dolan, L. (2000). TORNADO1 and TORNADO2 are required for the specification of radial and circumferential pattern in the Arabidopsis root. Developm. 127, 3385-3394.
Deblaere, R., Bytebier, B., De Greve, H., Deboeck, F., Schell, J., Van Montagu, M., and Leemans, J. (1985). Efficient octopine Ti plasmid-derived vectors for Agrobacterium-mediated gene transfer to plants. Nucl. Acids Res. 13, 4777-4787.
De Block, M., and Van Lijsebettens, M. (1998). GUS enzyme histochemistry on semi-thin sections of plastic-embedded Arabidopsis tissue. In Arabidopsis Protocols (Methods in Molecular Biology, Vol. 82), J. M. Martinez-Zapater, and J. Salinas (Eds.). Totowa, Humana Press, pp. 397-407 [0-89603-391-0].
De Jager, S. M., and Murray, J. A. H. (1999). Retinoblastoma proteins in plants. Plant Molec. Biol. 41, 295-299.
De Veylder, L., de Almeida Engler, J., Burssens, S., Manevski, A., Lescure, B., Van Montagu, M., Engler, G. and Inze, D. (1999). A new D-type cyclin of Arabidopsis thaliana expressed during lateral root primordia formation. Planta 208 (4): 453-462.
De Veylder, L., Beeckman, T., Beemster, G. T. S., Krols, L., Terras, F., Landrieu, I., Van Der Schueren, E., Maes, S., Naudts, M. and D. Inzé (2001). Functional analysis of cyclin-dependent kinase inhibitors of Arabidopsis. Plant Cell 13: 1653-1667.
De Veylder, L., Beeckman, T., Beemster, G. T. S., de Almeida-Engler, J., Ormenese, S., Maes. S., Naudts, M., Van Der Schueren, E., Jacqmard, A., Engler, G. and Inze, D. (2002). Control of proliferation, endoreduplication and differentiation by the Arabidopsis E2Fa/Dpa transcription factor. EMBO J. 15, 1360-1368.
Di Cristina, M., Sessa, G., Dolan, L., Linstead, P., Baima, P., Ruberti, I., and Morelli, G. (1996). The Arabidopsis Athb-10 (GLABRA2) is a HD-Zip protein required for regulation of root hair development. Plant J. 10, 393-402.
Di Laurenzio, L., Wysocka-Diller, J., Malamy, J. E., Pysh, L., Helariutta, Y., Freshour, G., Hahn, M. G., Feldmann, K. A., and Benfey, P. N. (1996). The SCARECROW gene regulates an asymmetric cell division that is essential for generating the radial organization of the Arabidopsis root. Cell 86, 423-433.
Dolan, L., Janmaat, K., Willemsen, V., Linstead, P., Poethig, S., Roberts, K., and Scheres, B. (1993). Cellular organization of the Arabidopsis thaliana root. Developm. 119, 71-84.
Dolan, L., Duckett, C. M., Grierson, C., Linstead, P., Schneider, K., Lawson, E., Dean, C., Poethig, S., and Roberts, K. (1994). Clonal relationships and cell patterning in the root epidermis of Arabidopsis. Developm. 120, 2465-2474.
Donnelly, P. M., Bonetta, D., Tsukaya, H., Dengler, R. E., and Dengler, N. G. (1999). Cell cycling and cell enlargement in developing leaves of Arabidopsis. Developm. Biol. 215, 407-419.
Edwards, D., Murray, J. A. H., and Smith, A. G. (1998). Multiple genes encoding the conserved CCAAT-box transcription factor complex are expressed in Arabidopsis. Plant Physiol. 117, 1015-1022.
Eshed, Y., Baum, S. F., Perea, J. V. and J. L. Bowman (2001). Establishment of polarity in lateral organs of plants. Current Biol. 11: 1251-1260.
Fellows, J., Erdjument-Bromage, H., Tempst, P., and Svejstrup, J. Q. (2000). The Elp2 subunit of Elongator and elongating RNA polymerase II holoenzyme is a WD40 repeat protein. J. Biol. Chem. 275, 12896-12899.
Fichtner, L., Frohloff, F., Bürkner, K., Larsen, M., Breunig, K. D., and Schaffrath, R. (2002). Molecular analysis of KTI12/TOT4, a Saccharomyces cerevisiae gene required for Kluyveromyces lactis zymocin action. Mol. Microbiol. 43, 783-791.
Frohloff, F., Fichtner, L., Jablonowski, D., Breunig, K. D., and Schaffrath, R. (2001). Saccharomyces cerevisiae Elongator mutations confer resistance to the Kluyveromyces lactis zymocin. EMBO J. 20, 1993-2003.
Gaudin, V., Lunness, P. A., Fobert, P. R., Towers, M., Riou-Khamlichi, C., Murray, J. A. H., Coen, E., and Doonan, J. H. (2000). The expression of D-cyclin genes defines distinct developmental zones in snapdragon apical meristems and is locally regulated by the Cycloidea gene. Plant Physiol. 122, 1137-1148.
Gietz, D., St. Jean, A., Woods, R. A. and Schiestl, R. H. (1992). Improved method for high efficiency transformation of intact yeast cells. Nucleic Acids Res. 20,1425
Goderis I. J. W. M., De Bolle, M. F. C., Francois, I. E. J. A., Wouters, P. F. J., Broekaert, W. F. and Cammue, B. P. A. (2002). A set of modular plant transformation vectors allowing flexible insertion of up to six expression units. Plant Mol. Biol. 50, 17-27.
Hawkes, N. A., Otero, G., Winkler, S., Marshall, N., Dahmus, M. E., Krappmann, D., Scheidereit, S., Thomas, C. L., Schiavo, G., Erdjument-Bromage, H., Tempst, P., Svejstrup, J. Q. (2002). Purification and characterization of the human Elongator complex. J. Biol. Chem. 277 (4), 3047-3052.
Hoffman, C. S. and Winston, F. (1987). A ten-minute DNA preparation from yeast efficiently releases autonomous plasmids for transformation of Escherichia coli. Gene 57, 267-272
Ikura, M. (2000). Calmodulin target database. http://calcium.oci.utoronto.ca.
Jefferson, R. A., Kavanagh, T. A. and Bevan, M. W. (1987). GUS fusions: β-glucuronidase as a sensitive and versatile gene fusion marker in higher plants. EMBO J. 6, 3901-3907.
Kaziro, Y., Itoh, H., Kozasa, T., Nakafuku, M., and Satoh, T. (1991). Structure and function of signal-transducing GTP-binding proteins. Annu. Rev. Biochem. 60, 349-400.
Kerstetter, R. A., Bollman, K., Taylor, R. A., Bomblies, K., and Poethig, R. S. (2001). KANADI regulates organ polarity in Arabidopsis. Nature 411, 706-709.
Kim, G.-T., Tsukaya, H., and Uchimiya (1998). The ROTUNDIFOLIA3 gene of Arabidopsis thaliana encodes a new member of the cytochrome p-450 family that is required for the regulated polar elongation of leaf cells. Genes & Dev. 12: 2381-2391.
Kim, G.-T., Shoda, K., Tsuge, T., Cho, K.-H., Uchimiya, H., Yokoyama, R., Nishitani, K., and Tsukaya, H. (2002a). The ANGUSTIFOLIA gene of Arabidopsis, a plant CtBP gene, regulates leaf-cell expansion, the arrangement of cortical microtubules in leaf cells and expression of a gene involved in cell-wall formation. EMBO J. 21, 1267-1279.
Koornneef, M. and Stam, P. (1987). Procedure for mapping by using F2 and F3 populations. Arabidopsis Inf Serv. 25, 35-40.
Krogan, N. J. and Greenblatt, J. F. (2001). Characterization of a six-subunit holo-Elongator complex required for the regulated expression of a group of genes in Saccharomyces cerevisiae. Mol. Cell. Biol. 21, 8203-8212.
Li, Y., Takasgi, Y, Jiang, Y., Tokunaga, M., Erdjument-Bromage, H., Tempst, P. and Komberg R. D. (2001). A multiprotein complex that interacts with RNA polymerase II Elongator. The Journal of Biological Chemistry, 276, 32, 29628-29631.
Liao, B., and Zielinski, R. E. (1995). Production of recombinant plant calmodulin and its use to detect calmodulin-binding proteins. Methods Cell Biol. 49, 481-494.
Long, J. A., Moan, E. I., Medford, J. I., and Barton, M. K. (1996). A member of the KNOTTED class of homeodomain proteins encoded by the STM gene of Arabidopsis. Nature 379, 66-69.
Masucci, J. D., and Schiefelbein, J. W. (1996). Hormones act downstream of TTG and GL2 to promote root hair outgrowth during epidermis development in the Arabidopsis root. Plant Cell 8, 1505-1517.
McConnell, J. R., Emery, J., Eshed, Y., Bao, N., Bowman, J., and K. Barton (2001). Role of PHABULOSA and PHA VOLUTA in determining radial patterning in shoots. Nature 411: 709-713.
Nelissen H, Clarke J, De Block M, De Block S, Vanderhaeghen R, Zielinski R, Dyer T, Lust S, Inze D, Van Lijsebettens M (2003). DRL1, a homolog of the yeast TOT4/KTI12 protein, has a function in meristem activity and organ formation in plants. Plant Cell 15: 1-17
O'Neil, K. T., and DeGrado, W. F. (1990). How calmodulin binds its targets: sequence independent recognition of amphiphilic α-helices. Trends Biochem. Sci. 15, 59-64.
Otero, G., Fellows, J., Li, Y, de Bizemont, T., Dirac, A. M. G., Gustafsson, C. M., Erdjument-Bromage, H., Tempst, P., and Svejstrup, J. Q. (1999). Elongator, a multisubunit component of a novel RNA polymerase II holoenzyme for transcriptional elongation. Molec. Cell 3, 109-118.
Pokholok, D. K., Hannett, N. M., and Young, R. A. (2002). Exchange of RNA polymerase II initiation and elongation factors during gene expression in vivo. Mol. Cell 9, 799-809.
Ponting, C. P. (2002). Novel domains and orthologues of eukaryotic transcription elongation factors. Nucleic Acids Res. 30, 3643-3652.
Pruitt, R. E., and Meyerowitz, E. M. (1986). Characterization of the genome of Arabidopsis thaliana. J. Mol. Biol. 187, 169-183.
Reinhardt, D., Mandel, T., and Kuhlemeier, C. (2000). Auxin regulates the initiation and radial position of plant lateral organs. Plant Cell 12, 507-518.
Rhoads, A. R., and Friedberg, F. (1997). Sequence motifs for calmodulin recognition. FASEB J. 11, 331-340.
Robles, P., and Micol, J. L. (2001). Genome-wide linkage analysis of Arabidopsis genes required for leaf development. Mol. Genet. Genomics 266, 12-19.
Rombauts, S., Déhais, P., Van Montagu, M. and Rouzé, P. (1999). PlantCARE, a plant cis-acting regulatory element database. Nucl. Acids Res. 27(1): 295-6.
Rychlik, W., Spencer, W. J., and Rhoads, R. E. (1990). Optimization of the annealing temperature for DNA amplification in vitro. Nucl. Acids Res. 18, 6409-6412.
Sabatini, S., Beis, D., Wolkenfelt, H., Murfett, J., Guilfoyle, T., Malamy, J., Benfey, P., Leyser, O., Bechtold, N., Weisbeek, P., and Scheres, B. (1999). An auxin-dependent distal organizer of pattern and polarity in the Arabidopsis root. Cell 99, 463-472.
Sanger, F., Nicklen, S., and Coulson, A. R. (1977). DNA sequencing with chain-terminating inhibitors. Proc. Natl. Acad. Sci. USA 74, 5463-5467.
Sawa, S., Watanabe, K., Goto, K., Kanaya, E., Morita, E. H., and Okada, K. (1999). FILAMENTOUS FLOWER, a meristem and organ identity gene of Arabidopsis, encodes a protein with a zinc finger and HMG-related domains. Genes & Developm. 13, 1079-1088.
Scheres, B., Di Laurenzio, L., Willemsen, V., Hauser, M.-T., Janmaat, K., Weisbeek, P., and Benfey, P. N. (1995). Mutations affecting the radial organization of the Arabidopsis root display specific defects throughout the embryonic axis. Developm. 121, 53-62.
Shaw, R. J., and Reines, D. (2000). Saccharomyces cerevisiae transcription elongation mutants are defective in PUR5 induction in response to nucleotide depletion. Mol. Cell. Biol. 20, 7427-7437.
Siegried, K. R., Eshed, Y., Baum, S. F., Otsuga, D., Drews, G. N. and J. Bowman (1999). Members of the YABBY gene family specify abaxial cell fate in Arabidopsis. Developm. 126: 4117-4128.
Smalle, J., Haegman, M., Kurepa, J., Van Montagu, M. and Van der Straeten, D. (1997). Ethylene can stimulate Arabidopsis hypocotyl elongation in the light. Proc. Natl. Acad. Sci. USA 94 (6): 2756-2761
Swinburne, J., Balcells, L., Scofield, S. R., Jones, J. D. G., and Coupland, G. (1992). Elevated levels of Activator transposase mRNA are associated with high frequencies of Dissociation excision in Arabidopsis. Plant Cell 4, 583-595.
Tsuge, T., Tsukaya, H., and H. Uchimiya (1996). Two independent and polarized processes of cell elongation regulate leaf blade expansion in Arabidopsis thaliana (L.) Heynh. Developm.
122: 1589-1600.
Uetz, P., Giot, L., Cagney, G., Mansfield, T. A., Judson, R. S., Knight, J. R., Lockshon, D., Narayan, V., Srinivasan, M., Pochart, P., Qureshi-Emili, A., Li, Y., Godwin, B., Conover, D., Kalbfleisch, T., Vijayadamodar, G., Yang, M., Johnston, M., Fields, S., and Rothberg, J. M. (2000). A comprehensive analysis of protein-protein interactions in Saccharomyces cerevisiae. Nature (London) 403, 623-627.
Ulmasov, T., Murfett, J., Hagen, G. and Guilfoyle, T. J. (1997). Aux/LAA proteins repress expression of reporter genes containing natural and highly active synthetic auxin responsive elements. Plant Cell 9: 1963-71.
Valvekens, D., Van Montagu, M., and Van Lijsebettens, M. (1988). Agrobacterium tumefaciens-mediated transformation of Arabidopsis thaliana root explants by using kanamycin selection. Proc. Natl. Acad. Sci. USA 85, 5536-5540.
Van den Berg, C., Willemsen, V., Hage, W., Weisbeek, P., and Scheres, B. (1995). Cell fate in the Arabidopsis root meristem determined by directional signaling. Nature 378, 62-65.
Van der Graaff, E., den Dulk-Ras, A., Hooykaas, P., and Keller, B. (2000). Activation tagging of the LEAF PETIOLE gene affects leaf petiole development in Arabidopsis thaliana. Developm. 127, 4971-4980.
Van der Straeten, D., Rodrigues-Pousada, R. A., Villarroel, R., Hanley, S., Goodman, H. M. and Van Montagu, M. (1992). PNAS 89(20): 9969-73
Van Haute, E., Joos, H., Maes, M., Warren, G., Van Montagu, M., and Schell, J. (1983). Intergeneric transfer and exchange recombination of restriction fragments cloned in pBR322: a novel strategy for the reversed genetics of the Ti plasmids of Agrobacterium tumefaciens. EMBO J. 2, 411-417.
Wada, T., Tachibana, T., Shimura, Y., and Okada, K. (1997). Epidermal cell differentiation in Arabidopsis determined by a Myb homolog CPC. Science 277, 1113-1116.
Walia, H., Chen, H. Y., Sun, J-M., Holth, L. T., and Davie, J. R. (1998). Histone acetylation is required to maintain the unfolded nucleosome structure associated with transcribing DNA. J. Biol. Chem. 273, 14516-14522.
Walker, A. R., Davison, P. A., Bolognesi-Winfield, A. C., Srinivasan, J. C. M., Blundell, T. L., Esch, J. J., Marks, M. D., and Gray, J. C. (1999). The TRANSPARENT TESTA GLABRA1 locus which regulates trichome differentiation and anthocyanin biosynthesis in Arabidopsis, encodes a WD40 repeat protein. Plant Cell 11, 1337-1349.
Winkler, G. S., Petrakis, T. G., Ethelberg, S., Tokunaga, M., Erdjument-Bromage, H., Tempst, P., and Svejstrup, J. Q. (2001). RNA polymerase II Elongator holoenzyme is composed of two discrete subcomplexes. J. Biol. Chem. 276, 32743-32749.
Winkler, G. S., Kristjuhan, A., Erdjument-Bromage, H., Tempst, P., and Svejstrup, J. Q. (2002). Elongator is a histone H3 and H4 acetyltransferase important for normal histone acetylation levels in vivo. Proc. Natl. Acad. Sci. USA 99, 3517-3522.
Wittschieben, B. O., Otero, G., de Bizemont, T., Fellows, J., Erdjument-Bromage, H., Ohba, R., Li, Y., Allis, C. D., Tempst, P., and Svejstrup, J. Q. (1999). A novel histone acetyltransferase is an integral subunit of elongating RNA polymerase II holoenzyme. Molec. Cell 4, 123-128.

1. An improved method for plant growth modulation of the type that modulates plant growth by the use of a gene, wherein the improvement comprises:
using a gene, or a functional fragment thereof, encoding a protein of the elongator complex.

2. The method according to claim 1 wherein said protein is selected from the group consisting of SEQ ID NO:2, SEQ ID NO:4, SEQ ID NO:6, SEQ ID NO:8, SEQ ID NO:10, SEQ ID NO:12, SEQ ID NO:14, and a protein having at least 40% similarity to any thereof.

3. The method according to claim 1, wherein the protein comprises SEQ ID NO:16.

4. The method according to claim 1, wherein plant growth is stimulated.

5. The method according to claim 4, wherein the growth stimulation is enhanced leaf growth.

6. The method according to claim 1, wherein said modulation involves altering cell pattern.

7. The method according to claim 6, wherein said cell pattern alternation is in leaf lamina.

8. The method according to claim 1, wherein said modulation changes plant architecture.

9. The method according to claim 1, further comprising:
altering expression of one or more other genes.

10. The method according to claim 9, wherein all genes having altered expression are selected from the group of genes encoding the proteins of SEQ ID NO:2, SEQ ID NO:4, SEQ ID NO:6, SEQ ID NO:8, SEQ ID NO:10, SEQ ID NO:12, SEQ ID NO:14, and a protein or proteins having at least 40% similarity to any therof.

11. The method according to claim 9, wherein said other gene encodes a protein involved in transcription regulation.

12. The method according to claim 9, wherein said other gene is retinoblastoma.

13. The method according to claim 1, wherein the functional fragment is the promoter of said gene.

14. The method according to claim 13, wherein said promoter comprises SEQ ID NO:15.

15. The method according to claim 1, wherein said functional fragment encodes SEQ ID NO:16.

16. A genetically transformed plant comprising:
one of more genetically modified genes encoding a protein of the elongator complex.

17. The genetically transformed plant of claim 16, wherein said protein is selected from the group consisting of SEQ ID NO:2, SEQ ID NO:4, SEQ ID NO:6, SEQ ID NO:8, SEQ ID NO:10, SEQ ID NO:12, SEQ ID NO:14, and a protein or proteins having at least 40% similarity to any thereof.

18. The genetically transformed plant of claim 16, wherein a modified gene is overexpressing the protein DRL1, or a protein with at least 40% similarity to DRL1.

19. The genetically transformed plant of claim 17, wherein a modified gene is overexpressing the protein DRL1, or a protein with at least 40% similarity to DRL1.


SEQUENCE LISTING (43 sequences)

SEQ ID NO: 1 - DNA, 2728 bp, Arabidopsis thaliana
  CDS (1303)..(2211)
gagctcttgaacatcttcagcgtcaaacgttcgcttgaaacccattcttaaaaaacctgtaaaaggagaaccaaagccattagttacttggcgtgaaggttatctgtgtgaaccacactcacactgacaagtgacaactataagtctatttaaccagttcagtaattaaattagagtataaagaaaaaatatttaaccccaattaatttaagtaaattaaaaaaaaaaatccccaaatttgaaaaccctaaccctagatttcaattcacggattaaatcgcgaaatcaagaatcgaaaatcaatgaatcggttaattatcatgaatacagacaaaagccctcgattaattgacagatctgacgaaccctagaacatcccaacctacgagcccagatcggataataaaattgggggaaactgaaattaagaacggatccgttcatacttttgaaggaaatcaacatcaaaaacaaacaattgatcagatctgttgttatcagatccagatctgactcaaaccctagatttgaccaaaacaaagggatgataacaaaacaggtatcaattggaagatcaaatccagaggaagaagaaaattacaatagatctgaagatcgatctggaagaaggaagggatctgagctgaaattcgaagaaaatttggtaggggaaaaaaataaaattgggggaattttcgaaagcggctacggttgaaaattcgtgcttttcgattaggggaaagacccgattttgaaacccggatttaactcggataccgagacaacatcgaatgagctgtactttatttaaattctttgaattacttgaatttaccaaattatcctcaccgtataccatcaacgtgcctagcaacttgtgttggcatatccggctcgtatctatcttctaaaccggacacaaattgatattcatatgaacaaaccggaccaagcgaattaaaccggttctaatctataagccctagattcccaaatcaaattttcacttttgctttcttcaacctgtcgacaacactcccgaatttttctctggtaatgtttttattaacttgatttttcgtcggaattgataattgtggaagtatgtactaataaagtttgatactttaacttgttctggggattgaatccgttatgatatgaatttctggaattttagtttttatatgggaatctaaagcttattcgtttgttatgtgttgatgttcttatgcacctaaagcttgaattgaaatagtcttgtattgattccatttttgtaggcagtgtgtttaaaaaccaatggcgctagttgtgatttgtgggcaaccttgtagtggtaagtcaatagctgcagtaactttagctgaaacattgaaagagtctgaaacgaaacagagtgttaggatcatcgatgaggcttcgtttcatctagaccgcaaccaaaactatgctaacatgcctgctgagaagaatctgagaggaaagcttaggtcagatgttgatagatcagtttcgacaggcgagattgttattgtagattccttgaatagtatcaaaggttacagatatgagctatggtgtatcgctcgtgctgctgggattaggtattgtgttgtttattgtgatgtagatgaagctcattgtaggcaatggaataaggaacggagtgatagaggtgaagatggatatgacgatggtatattcgaagatctggttagaagatttgagaaacctgagagaagaaaccgatgggattcacctctctttgagctatacccttctcgtgaagttatagacaaatcctctcctgtgattttagaggctgtgacttatctaaccaagactgttgactccaaaacccaagatgtgagaattctccaaccaagtattgccactcaggctgctagattttcagaagcaaattctctttacgagttggacagagcgacacaagaaatcatcaacgcgattgtggaacaacaatcactgggcggggctataagcagagttactcttggcaatgaactacctccgattgaaatctgcagaccgattgggctaccggagctaaggaggcttcgaagaacgtttgttaaattgatgggtcaatcgagtctaagcggtccaccgttaccaactgatgcagacagtgcaaagagacgttttgtggattacttgaacagagagtttggaggtaataacgcttgaaatttgttgggtgaaaaacatcttatttcttaaaggtttggttatgtattttcttaactcaagacttgttcttcttcatcgaaattcttttttggggctaaattgatgtgttataataggcccgttataataatgcaaacatattttggggctatagtatattttcttgtttttctttttttggttgtcgtattttcttgtttttctaacattgaaatttcaaaagacaacgacacgtttgacatttttggatgtatattaatttataataaatatgtcggcaaataaaaaattattcatttacaacaaatatgctttgatttgaaaataaccactacggtaattaaaagaaaacatacagcaacagttcttatctttgctaatcatattcttaattattttaaacagcttcattaatcgctaatgctttatttttgtcgacattcgttcatttaataattaattaatgcagcttacttttattcatacaaataatataaaatcataaaacgacgaa

SEQ ID NO: 2 - protein, 302 aa, Arabidopsis thaliana
MALVVICGQPCSGKSIAAVTLAETLKESETKQSVRIIDEASFHLDRNQNYANMPAEKNLRGKLRSDVDRSVSTGEIVIVDSLNSIKGYRYELWCIARAAGIRYCVVYCDVDEAHCRQWNKERSDRGEDGYDDGIFEDLVRRFEKPERRNRWDSPLFELYPSREVIDKSSPVILEAVTYLTKTVDSKTQDVRILQPSIATQAARFSEANSLYELDRATQEIINAIVEQQSLGGAISRVTLGNELPPIEICRPIGLPELRRLRRTFVKLMGQSSLSGPPLPTDADSAKRRFVDYLNREFGGNNA

SEQ ID NO: 3 - DNA, 5169 bp, Arabidopsis thaliana
  CDS (1554)..(1694)
tataagttggctgtaggataagaagaacccgaaatcagatttgtcatttcagaaaatggctttaagaattcacatatcaactctcctctactccaatgaccaactagtcaaacccgtttctgaccgtggatctatttaggcgtagtccattaaaaccatggtttagttgggctagaatttaattgtccacaacaatattggttgggctaagcccatggtccttttgaccaattgacagctctaataaaacctgtagataacaaaacaacacaagttttatatactataattaattgtcttctcaaagaacttaataggaagcaaaaaccatgtttgtttcatcccatttaaccaccttaaaatcgagatcaaacaattttacatatgcaagttcaacatacctccaaacatttagactcaagacgtctatccctacatacaaaatctccttgccctctttttttttgggtatgccaaaattttgaatatacgccaaactactttccaaatcgtttgcataacaaagatgaccataatctttttccttacgacgtttgtgaggtccactgtaatcatctggatcaagactctgatatagatatcttaaaccatatactatcgctaaaccatttagagtatcagtagtccatttattatttttaatggaccaaacacataaaattgagctgaaccgtatcaatccattaaaacttctgatatccaatattattataaataatactaaaaaactccttatgatactatatgaatatcacttatatacatgctcttatatggtgtgtagttttcatcccatccatgtagtcttctaaaggcgctgaccatgtcagtgatgacataagtccaacaaaggacatggttacgttggtctcgaatatactccgagacatctttgagcctttctctgatttcctcgtctagtagtagaaaattttctagtactcctgtctcctgatatcgtctaatatataataatatagttaaccttaaggcagtcaatcatcaatacgccatttatcctactaatttaactcatccaattggtgctacactattgacgtttgaaggttgggatgggtgtaacatatatcgcggacatgccttatccataaatacacttaaaattagtataaattttctgacttgccttacctgagtttgattattttcttagtaaagtattttgatattataatttggtagtaatataatcactataacatgcaagaactacaaaaccagaggtctctcaaatggacactcatttaattgttacgatagtaaatgtcacccatcaatgaaacctaagtcaattggatgatttgggcccaattggacctaattcggcccatttaatattccctcttttctaacctttgtttctcctcttgcttgctcacccttttccctgaatcttaagaattactaattcccggcgagttttcccggcggcggctagataatactggtttctgccagcaagatcgaaggagataaacttcgtaatcaaaaagtctccttctgataaaaagatgtcagaaaacacaaaagtcgaagcgaagagagtgttcataggagcaggatgcaacagagtcgtcaataacgtctcttggggagcttctggtcttgtctctttcggtgcccaaaacgccgttgctgttttctgccctaaggtctgtttgttccactcttgtctctctctttcaaaatcattccaaaagcaaagatgtgattttttttgggtgtgttttgtgtctttgtttaatgtttttttcagactgcccagattcttacaacgctcccaggtcataaagcttctgtgaattgcactcattggttacccacttccaagtttgccttcaaaggtaggacttaaagcttacttgctgtaattttgaatcctttggtttataggaacttcttttgctatctatttagagagttttaagtatgttgaatcgcttaggcattgctaaaggatgagacttttgatgttttataagatagagaacataggttgaagaatttgtgagtgatagcaaattgagaagatataggcattcaaattttctatttatagtctctatctacggtttggtgatttagttgctgtgaaaggttttatgtatttgtatctccctgaattttctatgatcgttcgcagccaagaaattggaccggcaatatctgctatcaggtgattcagatggtatcatcatcctctgggaactatctactcttaataacgatgtaaaattcccttactctcggtctgatcttagtttctttgcggtttctctattatctctgatccctagtatgttttctgtttgttttagcaacagtggagacatgtactacaactaccactgtctcacaagaaaggtgtaacttgtatcaccgcttatatggtttctgaaaccgatgcaatgtttgcttcagcttcttctgatggagttgtgaatgtctgggatgtttcttttccgtctcagcctagtggtaagcattaacgttgctgcagtatattaccggttaaacgaagtgaagtatgtcttctaagcagtatctacatctttttcttcctctgtgttatctgttgcagaggaatgtaaagtggtgtgtctggactctatctgtgttgacactaaagcaattgtaacgctttcattagcggaattgcctcaaaatcctggtcgatttgcccttgctttgggcggattggacaacaaaatcaaactttactctggagaaagaacaggaaaggtgcttcccgattcgcttttgtgccaaattattctcaactaaattcaattgtgagagtttttttggtttcatcctttttcagtttacttctgtttgtgagcttaaaggccacacggactggattcggagtttggacttctcattaccgttacacacaacagaagaaatacccaatagtattatgcttgtgagttcttcccaggacaaagttattcggatctggaaactagtgttagtcggtgatgttggctcgtggcgaagagaaatcactctagcttcttacattgaaggcccagttttcgtctcagggacatttacatatcagatttcagttgaatctgttctgattggacatgaagattgggtctattctgtggaatggcaacctccagtcatcgatttcattgatgggcggctagttaaccaccaaccgctgagcattttatcggcttcgatggacaaaacgatgatgatttggcggcctgagaagaaaacgggtgtgtgggtgaacgttgtctgtgttggagagctaagtcactgtgctcttggtttctacggtggtcattggagcccaaatagtctgtctattctggctcatggatatggcggagcttttcatctctggagaaatgttagtagcagcaaagagtctgagaattggcagatgcagaaagttccttcgggtcactttgctgctgtgactgatgtcacatgggctagaaccggcgagtacttgctatccgtgagccaagatcaggtcagggtttatggttgaggtgttaaaacgattcagtatattataatgcaaaaagtttcttttctgatatttcattctccaaatttgcgttttgcagaccactcgtgtgttctcagcatggaagaatgatgaggggaatgaagcagaggatgagcattggcatgaactggctcgtcctcaggttcacgggcatgatattaactgtgtggctatggttcaaggtaaaggcaaccaccgttttgttagtggagcagaggagaaagttgtgagagtgtttgaggctcctctgtcgttcttgaagactctgaaccacacttgtgcgggaggagaaggaagcttccctgaggatttacaagcagacgtgcaggttcttggagcgaatatgtctgctcttggattatcacagaaacctatctacttacactgtaagtgattttgattgatttaacaccattactagctttttaccaaaagcaatcttgagattcttaattttttttcgcagcttctagtgaaccactggagagaaatggcggaggcgaaggtttggatacatttgagacagttccagaggcggctccagcagagttgaaagagcctccaatcgaagatcagcttgcgtttcatactttatggccggaatctcataaactctatggtcatggtaatgaactattttctctctgctctgaccacaagggaaaccttgttgcttcatcgtgtaaggtaagttaatgcagttttcagaatttttttacacttcttcaatctcctagatttagattttagatctaactgagcaggattccattactaaattattatttttatatatgaaccacaaacttcttcaggctcaatctgcatccatggcagagatatggctatgggaggttgggacatggaaagctgttggtcgtttgcaatctcacagcttgacggtgacgcacttggagttctcttatgatgacactctgctcttgtctgtctcaagagatcgtcacttctccgttttctctatccaaaggacaggtaactcaagcggtttcaacttcaccgttctaaatcagattttctcaaaacatcgaaaaatgtctacgtttttagcgtctctaactcgtctgtttttttgcttctgttcagacaatggtgaggttagtcacaaactcatggcaaaggttgaagcacacaagaggatcatatgggcatgttcatggaacccgtttggccaccaatttgcaacttcatcaagggacaagactgtcaagatctggtcagttgagaacgacgctcgcattaaacagattcttgtcttgcctccgtttggaagcagtgtaacggccgtggcttggactggactagaccgcaacgagaagagtgggtgcgtggcagtggggatggaaagcgggctaatagagctgtcgaacgtaaagattatagaaacagaggaaggtacgactgcaacagcagctcttgctcttcggctcgaaccgttcatgtgtcatgtctcggctgtgaacaggttagcttggagaccaaccgagaagtgtgagtctaatcagagcttgcgatggttaacttcttgtggggatgataattgtgtgagagtttttaacttcaagttttga

SEQ ID NO: 4 - protein, 806 aa, Arabidopsis thaliana
  misc_feature: ELP2
MSENTKVEAKRVFIGAGCNRVVNNVSWGASGLVSFGAQNAVAVFCPKTAQILTTLPGHKASVNCTHWLPTSKFAFKAKKLDRQYLLSGDSDGIIILWELSTLNNDVKFPYSRSDLSFFAVSLLSLIPECKVVCLDSICVDTKAIVTLSLAELPQNPGRFALALGGLDNKIKLYSGERTGKFTSVCELKGHTDWIRSLDFSLPLHTTEEIPNSIMLVSSSQDKVIRIWKLVLVGDVGSWRREITLASYIEGPVFVSGTFTYQISVESVLIGHEDWVYSVEWQPPVIDFIDGRLVNHQPLSILSASMDKTMMIWRPEKKTGVWVNVVCVGELSHCALGFYGGHWSPNSLSILAHGYGGAFHLWRNVSSSKESENWQMQKVPSGHFAAVTDVTWARTGEYLLSVSQDTTRVFSAWKNDEGNEAEDEHWHELARPQVHGHDINCVAMVQGKGNHRFVSGAEEKVVRVFEAPLSFLKTLNHTCAGGEGSFPEDLQADVQVLGANMSALGLSQKPIYLHSSEPLERNGGGEGLDTFETVPEAAPAELKEPPIEDQLAFHTLWPESHKLYGHGNELFSLCSDHKGNLVASSCKAQSASMAEIWLWEVGTWKAVGRLQSHSLTVTHLEFSYDDTLLLSVSRDRHFSVFSIQRTDNGEVSHKLMAKVEAHKRIIWACSWNPFGHQFATSSRDKTVKIWSVENDARIKQILVLPPFGSSVTAVAWTGLDRNEKSGCVAVGMESGLIELSNVKIIETEEGTTATAALALRLEPFMCHVSAVNRLAWRPTEKCESNQSLRWLTSCGDDNCVRVFNFKF

SEQ ID NO: 5 - DNA, 5118 bp, Arabidopsis thaliana
  misc_feature: ELP3
atgaacggcgagctgaaaaagcaacctcggccaggtaaaggcggctatcagggccgtggattaactgaagaagaagctcgagttcgcgccatatcggagattgttagcaccatgattgagcgttcacaccgcaacgagaatgttgacctaaacgcaattaaaaccgccgcttgccggaaatacggcctagcacgtgcgcctaagctcgtggagatgattgctgcgcttcctgattcagagagagagactcttctcccgaagctccgtgccaaaccggttcgaacagcttcagggatcgccgttgtggcggttatgtcgaagcctcataggtgcccgcatatagctacgacggggaatatatgcgtttattgtcccggtggacctgactctgactttgagtatagtactcagtcttacactggatatgagcctaccagcatgcgagctattcgagccaggtgaagagtgtcttctgtgttatggaattgaaacttgtattacagcttctggattgatctagtaaagggtttggggtttaggtagttttgggtgaaatttgtgatttctgacccaatctaccaccggagtttgaacttagtttcagttgctgacaataggtggtagatagttttggggactatgttgtgtggatagcttcatgaaaacttcaattgagaactttgcttagggtcatagttagtttgtttgtgctttgatatttaggactcaaaatgattgctagaattcaaacattgtagattacaagaaatgtttatgaaatgagaaaaagatctagattatagatgagaatgtagacttgcttgataacacagctgctaaaaaatccataaggaaaatgctataagctggaaagtttgttcttctttatgagtgaattaaaaagacatagaattcttagattgaaattatggactcagtttgctgttcatgtgatgagaaggatgcacgtttcatgtttaaattgattttgctaaagccctgtttatggtcttaggtacaatccatatgttcaggcaagaagcaggatagatcagctgaagcggttgggtcacagtgtagataaggttagagtttgatatttctagtaacctgtgagtattattttcgttctttttaccagtctcttagcctagaggattactcctgattatatgaatgacaggttgagttcattttgatgggaggtactttcatgtcactgcctgctgagtatcgggatttcttcatacggaatcttcatgatgctttatcaggacacacttctgccaacgttgaagaggcagttgcttactctgaacatagtgcaactaaatgcattgggatgacaattgaaacgtgagttcgatgcagtaatcatgtacaacatctaaatttgaaatctgtcttttttgcctcaatgttttctctaaaagccttcatagtatctatatattgatttcattgtattctttgtggcactttgtggaaatggtcaagctgagtgaaattgtgaaaatatatataaaaatctaacaccaattgtttgtgtgaatcagtccttgttattttcattgatcattcattatggagtctagtctctaatctctgaagattcaaatatcagaatacattctctaatctgttttaaaaattcaccaggaggccagattactgccttggacctcatttacgacaaatgctgatttacggttgcacccggctagagataggtgtccagagcacatatgaagatgttgcccgtgacacaaatagaggtcatactgttgctgctgtagctgactgcttctgcttggctaaagatgctggtttcaaggtattcccttatagattagtctttataaatatttttagttttctcatgttagtatcaagtttatcttggcagctttcacctcctatgtttctgttcattaatatccttgaaaccaatgaacatttctttattttagagacactgctaaatttgtatccttacctccgtttttcattttgcctataggtggttgcacatatgatgcctgatcttcctaatgttggggttgagagagacatggaaagtttcaaggagtttttcgagagcccatcttttagagcagatgggttaaaaatatatcccacccttgtgatccgtggaactggactttatgaactatggaaaactgggaggtacgatactcttgtgtttaaagatgaatgcttgcatgtcacaacggaaaaggttaaatcatattgaaaatttctgcaggtaccgaaattatccacctgagcagcttgtggatatagttgcaaggattctctccatggtacctccatggacacgtgtatatagagttcagcgtgatattcctatgcctctggttacgtcaggggtagaaaaaggaaatcttcgtgaactggctctagccagaatggatgacttgggccttaaatgccgtgatgtccgtactcgtgaagctggaattcaggtcgcgatgtcaattgttgtttcttcctttgttttgttgttgtaaatactgaatttcctaacaaaagctagcttgtatggtctgagtacaggacattcatcataaaattaagccagaacaagtagagcttgtgcgtcgtgattacactgccaatgaaggttgggagacgttcctttcttatgaagatacacgccaggtacaagcttcatatctctaatcctcgcctctcacagcatagtccctaagactttattgaatcaattattatttggtttactaagatcacatacgtcttgtcactcattagatacattatgtcatgtttatcaaatatagatgttggtattctatttaaaaggaaaactgtcaagtttaggtaagacgctgagagcgttatcttttaacatatcagcatctgttcttggtcttttccgaatttctaagcctattggactaacagagtaagcatgtgcctttgtgtatcgttggatacagtgagtttgtaattttgatcaaatctccagtaagatctagtgtcatatatcatgtgatcagttttaaatccataatttcatttgcatgcactaaggcaagagtttcatgagaaacttgaaaagcttatcttctcaacccttaaacctgattggttactttggaatctgttaaggctctgaaattttcttaaacatttatctttgtggatgcaggacattcttgttggattgctacgtttgcgaaaatgcgggaagaatgtaacgtgtccagaactcatgggaaagtgttctgttgtccgtgagcttcatgtatacggaacagctgtaccagttcatggtcgagatgctgataagttgcaacatcaggtaccacccgagattcaacttgaaattgacacttaaattaaggacttgtgattctgattatcttattatttcctgtatcgaatttgcagggctatggtacacttctgatggaagaagcagagaggattgctagaagagaacatcgatctaacaaaatcggtgtgatttctggtgtaggaaccagacattactacagaaagttgggttatgaattggaaggtccttacatggtgaagcatcttctttgaaattatgtttacttaaaaaccaaattcgtaaaatctttatgttgaactagtgagattaatcccttgtggtatcattttgctctttaaattttcatagtcaaaaagttgagaaaacggctaattccttcacgaactatggcgattttgtaagattaatctgtaatgcgtacacatgttgctaagttttacagaattttgcatccaaaaacgcctgaactaaaatcatcattcatcacttccacttgtaagcaaaagatgttcacatcatctaaaataaaccttcgaaaactacaaattaaccgaagattgacttggctcttcgttgtagtctagatccatgaactcaaatccttcatctgatgtgttccgattcttcggtcttatatactccaccccaacattaccacacttcctcgtattgtgtccaattacaccacatctgccacaacgaacaatccttccctctctcgtaactttggttaccttcactggaggagactcttgcacttcctttttccttttaggattcttacttctctcctcatgaaactccacagaagaatcagggcttgagctaacaaagaaaatccaagacaagatcgatatcaaagtgtcaaaagaaacgctagaaacgcagccatattgttagaaatacttactccataaactccaaatgatgatagtcttgtacccttgataactgttcatcatgttctttttgtatgatctccaacgctacttgctttgcctttcgacattgatcacgcgatgcaatcaaattccaacgctgctggatttgtccttgaatagcttttggtttgaacttaggattactccttatatcgtttagaaacagcctagcaataacagcttgagtcaatagcttggtatatccattcttgacacaacgatgctcgtcgtgatacacttttatcatccactttccaaccggttcttcgaaagaacaataaattctccacgggcaatctcctttcatcgcacatttgagctctgatttatcttttccccatctcgtaaacttcacattccatcctcctttcaacacgtaatccaccatagcttctttaaactcttccaaagtgtcaaacacctgttttatttttagcttgccactacgcctttcatagtgttctgttttaatatcggaactaacaggtgttgtatctcggtaatcatcagcattcttctcttcctcaccaaatccagtgatataacattcaactagattttcttcatcaaactcataatcataatgaataactgtctcatcttcaacgttatgagaagcatcttcacttgatttatcattcacaccatcatcaatatcagaactacaaagttaaacaaaaacagaaactcgtttaggcatttaatcaaacacatggcatgcatataaaatttaaccctgaaagactaaattgttccatacaatctccagccctaaacgaaaagttcgacataaaccctactctagatacttgcataacacaatttcctctattc

SEQ ID NO: 6 - protein, 559 aa, Arabidopsis thaliana
  misc_feature: ELP3
MNGELKKQPRPGKGGYQGRGLTEEEARVRAISEIVSTMIERSHRNENVDLNAIKTAACRKYGLARAPKLVEMIAALPDSERETLLPKLRAKPVRTASGIAVVAVMSKPHRCPHIATTGNICVYCPGGPDSDFEYSTQSYTGYEPTSMRAIRARYNPYVQARSRIDQLKRLGHSVDKVEFILMGGTFMSLPAEYRDFFIRNLHDALSGHTSANVEEAVAYSEHSATKCIGMTIETRPDYCLGPHLRQMLIYGCTRLEIGVQSTYEDVARDTNRGHTVAAVADCFCLAKDAGFKVVAHMMPDLPNVGVERDMESFKEFFESPSFRADGLKIYPTLVIRGTGLYELWKTGRYRNYPPEQLVDIVARILSMVPPWTRVYRVQRDIPMPLVTSGVEKGNLRELALARMDDLGLKCRDVRTREAGIQDIHHKIKPEQVELVRRDYTANEGWETFLSYEDTRQDILVGLLRLRKCGKNVTCPELMGKCSVVRELHVYGTAVPVHGRDADKLQHQGYGTLLMEEAERIARREHRSNKIGVISGVGTRHYYRKLGYELEGPYMVKHLL

SEQ ID NO: 7 - DNA, 3657 bp, Arabidopsis thaliana
  misc_feature: ELP4
tatggctgcaccaaacgttcgtagtagtagcagcttctctcgcaacatatcagttgtgtcatcgcctcaaattcctggtctcaaaagtggtcctaatggcacagcttttatatcgtcagggattcgtgatcttgacagtgagctttctcctttttatttacacatcaagtctcatgaggataagagagtctggttttggggctaaaaggtttgtattttattacttttccagggatactaggtggtgggtatcctttgggaagcttagtaatggtaatggaagatcctgaagcaccgcatcatatggatctgttaagaacttacatgtctcaggggcttgttaacaatcaaccacttctttatgctagtccttcaaaagatccaaaagggtttcttggtactttgccgcatcctgcatcatccaaagaggataagcctactgcgcccgaccctgatcaggtgctactttcttgtctgtaccttcgttctgtttgcctttttcctggctagttaaaaattgggtagtgtgcacttttgtcaattctgtagggagagagcttgaggattgcttggcagtatcggaagtatttggaaaaccagaagaatgctattggtaatttagttagtgcggttatatattcatttgcatatcattcaagaataaattgagctactagtaagttagatatcattctttggaaacataatcctctttacctattctggatcagatgattactccaatgactttgatatgagaaagcccttggagaggcaatttctgagcggacggcccatagattgtgtcagtctgttagactcttcagatctttccattgctcaggaccactgtgctacgtttttatcaaaatttccaaggttgtgtggcatcctttcttatttgatctcaattactcgctctagtttcgaatcaataaattttgttggtcatacctatgcagcaaattctgctcaagctcaattctttttacatctctcttatacttccaagctcagtttgtttatattgttttagtttgttactgagtcagttattgttcaaattcttctcttatttactggctttgttaagtagattgtcctttttttcttaagaacctgtgtattgtttcccacagaaacagcagtaacattgcatctattggccgcatcgcaatccagtctttttgctctcccctgtgtgagtattctgaaaaggtaattaggagagttttctggaacttttagcctatatcaagtcaacaatgctttctaacgaaaattctcttgactctatagttaatgatagctgaagcttgtaccagtatcacgtcgttatgtattatattgttgattttttgacattttgtttctatcaacacaggaatcagacatgctctcattcataagattgctgaaaagtatgcttatggtttcaaatgcggtagccattgttactttcccgccttccctgctctccccatcttcctctaaaagactgcagcatatggcagataccttgctctctatcaaagcaatcccaggtttgctttccctgattttcttattcataaatagattatcatagttgtttcttgacaagtatccttgcctttttttccagatggtgacaaggaattggagaaactcctcactggctacaaggatataaatggattcctcaacatacacaaggttgctcgtatcaacacacaggtatttacaaattgtgtaattctcaatttatatagggatattattaccctgaaacctgtaagcaagaacccctttgtattttgccaagtcaaaaaaacatattcaatggataaaagtctgatgaaattcaaaatgtctactgaacaacttatccagaaatccccatctaatccatttttctctcttttgatgagaatgacatgaacttgcttagaaaccgttgaaaaaatggattcactatggtcctaaaccactggtttgatctgaattgtttaatggaacaggtgcctgtaattcttgaggcaaaaaccttctcaatgagcttgaagaagagaagattcttggctctagagtgtctgaatcaagcccctgtagacgggtccagtggaacatcgtatggcacatctggtagctgctcatccaaatccggagcactagatttttgagttgctgagttagtctttcttttcttcttcttagtttttttttttccctctccattgagttctctaagttcactcactaacctgagcagttacattattattatagtctttggtcttaaaaacagttttgacagcaatatgttttgtttttataatgaaatttatttcatttctgtattatatatactcttatgaaactatgaccaatgatagatgctagttactaaacgttcaatgtaatcacttcaacatgttcacgtggttatttgttgaaccatccatatatataacatataaagaaagaaggtgagacattgaatgaagagttcaaaaatatggttgatctatatttggtgctcctttaattcagtgttcctttacttaccaaccaacgaccatatcatttgaattattgctcataaatctctctctctacccaatctctttatttagtaccctattcttcttgactttgtttctttccaaagcttatacaagagatattcattcttaaactaagattttcatatatattgaaaaagaaagaaaagatttctcatcattttactttttggtagagagcatttcaagtaaaaaatactttgagaaacaaaaagagaaaactagtatgagaaacaaatccttaaaaaaggaaataacagtaatgcacgtgatgatataacaaaaggttttgttttgtttgtttttttattgtttttttcttcttttatttgtctgttataataatcaaaaaacaagaagaaaaaacgagacgttcatgcatttaatctcgcaccttacgacaaagagtgagagagaggatcgctcaggtttgttcatcttcaccggacgattagcttttgcggtgggagaaagaacggcctgtcttcagctctgaaacagaggaacagagaggttaggaaggagacaatggttggacccgcgcggcctcagatcgttttgtttggatcttccattgttcagatgagctttggccatggtggttggggcgccattctttccgaggtctacgctcgtaaggtatcgtcagattcatcaatctctctctctctgtggcttctggattcgatattcatccttggttagattactagagcgttgattctacaagtagatcgcttattctaattactctgtattatttattattttccctaactatacatttgttcattgaaattcgtcttttgttttacgattgtaagcgtgtattatgcgaaccataatgttatttctccatttggatgttatgtgtataggccgacatcattctgcgaggatattatggatggaactcttctcgtgctttggaagttgtcgaccaagtgttccccaaggtactctttcaactccctttttttttgcttcttctctctttagactattagtttcttcaatactagattatgataaagattcagcgttttataggtctttatattagtccgtataggttgatgtgtgcttatcattt

SEQ ID NO: 8 - protein, 355 aa, Arabidopsis thaliana
  misc_feature: ELP4
MAAPNVRSSSSFSRNISVVSSPQIPGLKSGPNGTAFISSGIRDLDRILGGGYPLGSLVMVMEDPEAPHHMDLLRTYMSQGLVNNQPLLYASPSKDPKGFLGTLPHPASSKEDKPTAPDPDQGESLRIAWQYRKYLENQKNAIDDYSNDFDMRKPLERQFLSGRPIDCVSLLDSSDLSIAQDHCATFLSKFPRNSSNIASIGRIAIQSFCSPLCEYSEKESDMLSFIRLLKSMLMVSNAVAIVTFPPSLLSPSSSKRLQHMADTLLSIKAIPDGDKELEKLLTGYKDINGFLNIHKVARINTQVPVILEAKTFSMSLKKRRFLALECLNQAPVDGSSGTSYGTSGSCSSKSGALDF

SEQ ID NO: 9 - DNA, 13000 bp, Arabidopsis thaliana
  misc_feature: AtELP1
accacattgtcacttgcgttctcgcgttccactcgtactgtacacagagaaacaaagtgagcaagtaatctacttcttctacccgcacgttccaagaacaagaatgatgttacacctgtttcctttcatcaccgtttttggccaacttctttgcactctccagccatgtccccagaaggcaattatcatccgaagcaagtagaacgtccatgtcctttataagctccagaaacttctcactcaattgtcctaagcttccgatatctttctttacaaaagctgtcactgcttcggtgtacacctggtttgctagcttcgataaaacttgacgcgtcaaatcaaccatgtcatacctgcaagcaaaacccaaaggaatctgtaaccactaaaatggtagataatctatctctacttgcatctacctatatgttaagctccttgacagatcatccccagcttccaggaaaagtttgagagcttggatcacctcctttgtagaataccataaatgggccttaggcaaatcagcggtcttatcttgaaacaagactcttctttttgtttcatatgggccagtggagatcatatatgaatctttttgtttcaagtcgtcttgtacagaactagatggatcccagtccggaagcttgactatgaaatctgtgttatggtcctaaaacagagaattttaaaagcaagtttctaatgaattatagagcagatctaaggagagatgctgcggtagtaaccaaaacaaaaaaacgtactgcgattccatcagtgcaattgtagacagtgtgatacagaatctcccaagcggcctcgatttgatgattttctttcatgtatcttctgcgtgcatagctcttcaaccatttctatgtacacgaatattaaaaatgtatgaaactgattgtgcaaacactttcaagtgtcacagcaagggtgcatactatatatgcattacctgtacatcaactttctcatctcgaaatgccatttcagaagttagttcgtaaaccaccgggttctgctctattccttccatgcacattcctacaccgacctatgtgcaacataaataacaagattccatttctaagcggagaaaatagtctcacctatagacataagataaaaaatgtgaaacatattgtgaatgtcgtagttatgagttaccatggttgaatttttgctaacacgagcatcaactggtcctgaagaaattgaatcaagagcaccatacatttcaatgttgcctccaaaattgtgaagcatgcacctggaattcatccgattataataaaaacacgtaaaaatacaactgactcaggcataagaaggaaactgagacaacaatataccagatataaggagttccataaaattgggcggacttgttccatatcggtttgacttcagcatataggtcaagaactatcattttgccaaagggaactgaatgtaagagtgcctgcagaaagcaagcgaggctgtgatattgaatcaatcagcattcatcatatacactaaattggatgtttactttcaactgaggcggcttccagaatttggagtctgaactaaaaagccaaccctgcaaaaaggtgagtctcaagtaagaaaacattcataaatagagaaatctctgccccgtcattctagattgttgttacttcaatactaaaatctgtgtctgtgtgtttacagttttctaaagtctaaaaagcatcctaaagtacatacgccactgagaataaaatggaaaagaaggcatacttgcattaaccatacagcattcttgtttccttttgacatagctttgtacactgcagctcctagtgaagatatatattcaggttcacttgtgggaggagtattctcattgaatgtatcactgcattaacacaagtaagattcgtatcatcattccaaacaaagagagttgatttttcttgaatcgtgcatttttgcggaaggttgttttcaccaatacctgcgtccaggatactggaaaaacttgtaagaaataatttacctcttaagtgcaaattgaagataattaccagttatatatgtttgtaatctctccatattctgagacagtaacaaggtaaaatcaaagacatataaacgtgtaattatatcgaaaagactgtgatcaggtgggagctttgttagtatgtatctaatttgagaaaatgctttcaatggatcacacaatttgccaagattaaaaaaaaagaaatagcttcagggtaccttcagtttgttgtttaataaaagcctctccaatttcaatgaagagagggtcagagggattcagaaggtaagtgcagcaccagcgagaatcaccatccaccgtgttcctgagaatacacaaatggtaacggtaaggttattgtcaatgtgaagacaaggcagtaaaaaaatagcatttttttctcatcacacaactcaaaaagtttcatccattggattgaaaaaagtttccggacatgatttacggaaggaacacataccagttatcaagtcgagttatatttgcctcaggatatatctttctcaaagctgatggaacattgcctgagaatgatgggagtactaggaaaggaggaaacaaaagaaaatataagatgctatgagaacaatcaatcgagtatatgaaaagagaagaaagtgattgttcttggacctggagtcatgccaaacttcaacatccgagaaagtatctgtttctgcaaaagcaactggtcatctaaccagtttttcgataatggaccaccccaactgtgatgtagatgtagttagagatatgaattatgtgagcttcaagaatgaaagataaatcaagctcagaaggagcactggagataggagcctgaaaatatcagagatcaggttatttacgcgtgaagatttcccatgcgagcccaagcaagaaacgctggtccaccaaaataatcatccagatcttctttgctgatattgaacctctaaacaaaattgatatagaacttggttagcggtatatatcaacttatattaaataaaaaagttgcaatatctaatcatgtcacagcaaacaattactaatgcaatctgactttttaccttgaaaaccttttgccaaatagcttcttgtccagtgaatgctagaggcaggttaattccttgcaatgccatccagtcaatctctctctcccatctctcccatccccaccaaacatatgaatctggaaaaaacaagaaagcaggcagtagcaggagtgtgattctagtgaatcatgacatgaggaaacttttgattccaagaagtgtatatatcttacagctagatgtaacaacattttggtaatagttccaagggacaggacgccggataaagatgcgtttagaatctatacgaggtagatgtccaggttgtggaacagaggcaacctgaattccaccagttttgtcccaagaaacatgagcattacatttatacttcaaataccaatggagcccagaagcaatctcaactccagttgtgcctttaatcctgagacacaagatggcaaaactaaaagagaagaattttaagtaaaattcgatccctcatcaggaaatcagaagatacttacagaatttcaggtccaattcgtcccgggccatcatagttttcaattacaaagcagctggtaccaccacaagcatcctattgtaacaaaaaacaatcagtactgaagaatcacctagcatagtggggcctaaggaaataaaaggaagaacctttgagataattctgagctcaaagctctgagaatgggtgggaagcagtctctgcaataaaccctttgctgctgattcttgtacagaagaagttggtaataaagaatccaaacggtcaagcagaccatcaatagttggatgatgttttgacacagtttgagagtgaaaagaaattatcaacaaaaccaacaaaaccaatttgatggaatgcatgatgatgcttttcctcactagtgagagaatcagcaaggaaaggcatagaaaactgatctctgaagaaagtggccggataataatattatatttcttacaaactaaaccggtataaaccaagataaccggtgtggtaaaataaaccgggtcagtttttcaagtttatagcaaaacgaaagagaggaaacagagaagaaaagaaaaacagagaagaaggcgtgttttaggttaaaccctaaatattattatatcagtgatttctggggtttaaatctgcttcctggatctgtagctgcaaagttcgttgatgttgtactgattcttacttcttcgaaaatgaaaaatttgaagcttttctcggaggttcctcagaacatccaattgcattcgactgaagaagttgtacagttcgcagcttatgacatcgaccaaagtcgcttatttttcgcatcttctgctaatttcgtttacgcccttcagctctcttccttccaagtaagtgaattgctttctgtgagagaattcgtttcctccttggttaatcctgtttctgcttaataaggaaacgagtgagaaaaaagttggggactttggtctaaagatggatttttctttttattttgcttttgattcaagtttcttgtggatgaacttgcaagtgaacttagcgagtagggtctgtttaaatcgaaagtctttctctttatgtttatgtatttcttgtggcagaatgaaagcgctggggcaaagagtgctatgcctgtggaggtctgcagtattgatattgagcctggggatttcatcacggctttcgattatctcgcggagaaggagtctctattaattgggacttctcatgggcttttgctagtgcataatgtggagagtgatgtgactgagctagttggaaatatagaaggaggtgtcaagtgtatctcgcctaatccgactggagatctacttggattgattactggtcttgggcagttgcttgtaatgacttatgattgggctttgatgtacgagaaggcgcttggagaggttcctgaaggtggttacgtacgtaaggaccccttaccttcttgtcatattgactgcataagggttaaatttatgggtttgagttgaaacttatcgtgaacagcttttgtttatttgctaaccatctgaattaaatactgttttaggtgaaacgaatgatttgtctgtaaattgtgggggtatttccatttcctggaggggtgatggaaagtattttgcgactatgggtgaggtttatgagtctggttgtatgtctaagaagattaagatatgggaaagtgattccggtgctttgcagtcttcttcagaaacaaaagaatttacgcaggggattcttgaatggatgccgagtggtgctaaaatcgcagctgtttataagaggaagtcagatgatagcagtccatcgattgctttctttgagaggaatggacttgagagaagctcgtttagaattggtgaaccagaagatgctacggaatcatgtgaaaatctgaaatggaattctgcatcagatcttcttgcaggggttgttagttgtaaaacatatgatgccattagggtgtggttctttagcaacaaccactggtacctgaaacaggagattaggtatccaagggaagctggggtaacagtcatgtgggatccaacaaagccactgcagttaatatgttggactctgtcagggcaggtgtctgttcgccactttatgtgggtaacagcggtcatggaggactcaacagcctttgtcatagataactccaagatactcgtgacaccactatctttgtccttgatgccacctcctatgtatttgttcagccttagtttttcttctgctgtcagagatatagcatattactcaaggaattctaagaattgtctggctgtgtttttgtcagatggaaacctgtcttttgttgagttccctgcacctaatacttgggaagatctcgaaggcaaagacttcagtgtggaaatttcagactgtaaaacagcactgggctcttttgtacatcttctatggttggatgtccactcacttctgtgtgtctccgcttatggttctagccacaacaagtgtctctcttcgggagggtatgacacagagcttcatggttcttatttacaagaagttgaagtagtctgtcatgaggatcatgttcctgatcaagtgacttgctctggctttaaggctagcattactttccagacacttctcgaatcaccggtcctggctcttgcctggaatcctagtaagcgtgattcagcctttgtggagtttgagggtgggaaagtgcttggatatgcgtcaaggtcagaaattatggaaactcgtagtagtgatgatagtgtatgttttccatcgacttgtccttgggtgagggtggcacaggttgatgctagtggggtgcataaacccttgatatgtgggcttgatgatatgggtagactgtccatcaacgggaagaacctatgtaataactgtagcagtttctcattttattcagagttggccaatgaagtagttacccatctgattattttgaccaaacaggattttctctttattgtcgataccaaggatgtactgaatggagatgtggcacttggaaatgtgttctttgttattgatggtagaaggcgagacgaggaaaatatgagctatgttaatatttgggaaagaggtgcaaaagtgataggagtcctcaatggagatgaagctgctgtgatattacaaactatgcgggggaatctcgaatgcatatatcccagaaagctggtcctctcttccattacgaatgcgttagctcagcaacggttcaaagatgcatttaacttggtaaggcgtcatagaatcgactttaatgttatcgtggatctatatgggtggcaggcatttctacagtcagccgtagcatttgttgagcaagtaaacaatttgaaccatgttacagaatttgtttgtgccatgaagaatgaagatgttacagaaacgctgtacaaaaagttttccttttccaaaaagggagacgaggtttttcgtgtgaaagatagttgcagtaacaaagtatcatcagttctacaggcaattaggaaggcccttgaagaacatatcccagaaagtccatcaagggaactctgtattttgaccactcttgcccgaagtgatccaccagctatcgaagaatctcttttgaggataaaatctgttcgtgaaatggagttgctaaactcttctgatgatataaggaaaaagtcttgtccatctgcagaagaagccttgaaacatcttctgtggttattggattctgaggctgtctttgaagctgccttaggtctttatgatctgaaccttgcagctattgtggcactcaattcccagcgtgatccaaaagaatttttaccttatctccaggagctggaaaaaatgcccgaatccctgatgcatttcaagattgacattaaattgcagcgttttgatagtgctcttaggaacattgtatcagcaggggttggctactttcctgattgcatgaacttaataaagaaaaaccctcaacttttccctctgggcctcctgctaattactgatcctgagaagaaactagttgttctagaggcttgggcagatcacctcattgatgaaaaacgttttgaggatgctgctactacatacctatgttgctgtaaactggaaaaggcttcaaaggcatatcgtgaatgtggtgactggagtggggtactccgagtgggggcgctaatgaaattaggtaaagatgagatcttgaaattggcatatgaactctgtgaagaggtcaatgctcttgggaaaccagcggaagcagcaaaaatagcgcttgagtattgcagcgatataagcggtggaattagtttgcttatcaatgcgagggaatgggaagaggctttaagggttgcatttttgcatacagcagatgatagaatctcagtggtgaaaagttctgctttggagtgtgctagtggtctagtaagtgaattcaaagaatcaatagaaaaggtaggaaaatatttgactcgctatctagctgtccggcagagacgattattgcttgcagcaaagctcaaatccgaggaacgatctgttgttgatcttgacgatgacacagcttcagaagcaagtagcaacctgagtggaatgagcgcctatactttggggtacatccttgcatttgtcttactctcgcagtctttatttatgtggaatttttctcagctgatcttacagatttcaatctaaaacagagctgaattaggtattgatcgtttttagaaacaggagctcatgttcgttttttttgtctcatgacttcaacatgttttctgattgtctcctcctcgattaaccagcatctgattatatgcatgaacattccaacatatataattttgtattggtaaagtgataagtttccgggtgggactttagtgcatgattgtttagcctgctagttgagagctgacgattcaaaaggagatttaggcacgttctgcattttctttggttttgattaagcatttgaagggacaagcgtgttgacaagtcgtaacgtaaaagttgtagggcattgggtagagaatgaagacttttctcaggaatcaatttctctataatgtagttgttttgtgggttaaatagggaaacagttactaactagtttggaccttaacttacaagttcttgtttcctctatgctattattccaggacaaggagaggttctgctgcttcagtcagctcaagcaacgctactagcagggcaagagatttgaggcgccagagaaagagtgggaaaatccgtgctggcaggtatatatatagacatctcttatgcaaattgataggtaccaatgatgagcaaaaatgtaaatgaatttggggtaaaaatgcagtgcgggtgaggagatggctcttgttgatcatttgaaaggtatgcgtatgaccgatggaggaaagcgagagctgaagtcgttgttgatatgtctggtaacacttggtgaaatggagtctgcacagaagttgcaacagactgcagagaatttccaggtctcccaagtagcagctgtagagctagcacacgatacagtgtccagcgagagcgtagatgaagaagtttattgctttgaacgttacgctcagaagacgagatccacagcacgagattcagatgcctttagctggatgcttaaggtcttcataagcccatgaaaatctctacatctgtttttgactgaataagcttgtgctttttaaacatcaaaaccttcctctaccgttttgtatctttcctattgttactgtactatgagattaaggtgcatctgctacatttttgttcattgctaatgcttacattccgattcaaaaggaacctttaaccacaacaagtaaaaaatgacgagatgtccgttacacagagctcacatgtcgtttcgactcaacagaatcctttaaccacaatggcaaaccagtagcaccattattttaaaagtgaaattatttttctattaaataaacacttaggctagtgaaaggtttagatcgcctagatattaacacaaaagaaagaaagagacaaaagaaacaaagcaatcataccaccgatgcagtgcataaaccagcctaaagtaagtgaaacggtatattaaaaactaaaaaggacaaaaaggcttcttatctcttgcatagaagacccaagatctttatcaacaaacaaaaaaagaagaagaagacccaagacgtgtcatttgttttactagtcgccactccttttctcaaataagagtctccgcaaatggaccatggtcaactccatgattcacaacaaaaaatcgaactccaatagtactgttcatcaaacagcatcatccataatcaatcaaccgcatgtcatcgttgatacgcatgtacgtaacgtggtgtgttattttttgtaggtgtcaaacaacatggtctgtttatgtgctggtacaactaaaataaatatgataaatgtgatgagccgaggatatatttatataagagttcaaagtaggtttataatatatttctatgaaacttattgctgtcactcgaggaggggtgaagaaaccatatatgtattttcaaataaccatacttttgttttgtaatatatgttgtgtgttgcaaatcattttggttatccacttcatattagtttgtctcagtttcttgttttgctttgtgacgtaaaatggtttcattatgcagtttgtatatgttaggtccctatatcctatcgagatagtaatgttttatataatagagaaaaatccattagaagcagttgataaagtgaagtgactcatatgtagatcggtttagacgattaacaaaatatgtgacgaataaaataaaataaaaaacaaaggtcatataatgagcggaagctagtgagtaaagtacttgaaactgccaaattaataataataaaagtgggtatgttgacaaaaaaattaataaaagaggtctatgagaatcagcacccaaccaatcaagaaatgtttaccccttaaaacacgagtttagtgctctttttctttcctcttttttaggcaaatgtcttaaattccttgcacttcaacacaatgctttttacttctgaacacatgaaaacgttatgaggcttcaaaactgaatctatgtataaataatacacacgcatatatttaactttgttttttcttttcttttccatataacaaaccaaacactactcttttctaaccaagagaaaggttttatatatgcagaatgaagtttgagagagcaaggccattcatagccattgttttcatacaatgtttgtacgctttgatgtctatagtagcaaaattagctttgaacaagggaatgagccctcacgtcttagttgcttaccgaatggcagttgcttccgctctcattacgcctttcgctctcatcctcgagaggtttcttttcttcttccggtccatgcaaggcctgtaacacaaaaacatacactttgaggttaaaataatggttccttttgctccaaatgcaggaacacaagaccaaagttgaccttcaagattttgttacaaatagccatccttagtttatttgagtaagagtttgtttaaatcatttctgatgcacaatgatagtctgttgtaacttgatttaaatgattaaacacaggcctgtggtcgaacaaaacctgtactactcagggatgaaacttaccactgccacattcacatcagctttgtgcaatgctcttcctgctatgacattcataatggcctgcgttttcaagtaactctctcactctctagaccgatattatttgctgatgcacacatcattgcagtactgaaacaatttgactcggtttctgtttcttgaaaggctcgagaaggtgacaatcgaaagacgccacagccaggcaaagctggtgggaaccatggtggctattgggggagcaatgctcatgacattcgtgaaagggaatgtcattgagctgccttggacaagcaactcaaggggtcttaatgggcacacacacgctatgaggattccaaagcaggcggatatagcaagaggatcaattatgcttgtagcaagctgcttcagttggtcatgttacatcattttacaggtatgatccattcccgcttccatgttcatgaatcttggtagctaacctaaagccagaagttttccaccagttttactaaagcttaacatatttctcgagataaaacattgagaatatatctctagtgaacaaaaatgtgatatcacagagtacttatggttacagaggactttatgtttttctcaggcaaagattctggctcaatataaagctgagctctcgctaacagctcttatgtgcattatggggatgttggaggctactgttatgggattgatatgggaaaggaaaaacatgtcggtctggaaaatcaacccggacgtgacgcttttagcttcaatctacggagtacaacaattcttgtaatctgttcctacattttccagtttgtgtgtatgttttgctgagagaagaagtattgttgattgtactacagggactagtctcagggttagcatactatgttattggatgggcgtcaaaggagagagggcctgtgtttgtgagcgcgtttaacccgctcagcatggttctcgttgctattctgagcacctttgttttcttggaaaaagtgtacgtaggaaggtaataataaccttgatctaagactactgttgtatccaaagttagaaaaattgattttcactaaatgagcttctaaatttgtagggttattgggtcagttgtcattgtcattgggatatatttggtattatggggtaaaagcaaggacaaaggagggatgcttcaaccaaacgctggatgcgcagagactgtggtaaagattgaccaacagaaggttccgacgcctgataataaccaagtagtatctatcagttaccatttaatgatcccgaaggcagcagctcggtctcaagagtgagtctaagatgccgattctgttccaggatctctatatatcgcaaaaaaacatttttgatagtattggtttgccagaataagagcaagcaatgcgctacattaagtctcataaaagctaagaacttggagttcttgcagtagattatatactttagtaagagacctagacttcacccagacttcacttacgacaaaaagcctgccagttgataactcgtcttctcaagatgccaaagtgacatcatccttcccatagatacgctgaagttcaagtctagcaacgtgatatgaagctgaaaagagacaaaattagaacaatgattggcttttgtatcttgttgtcatgctctaagcggctatatcctataaaagtttctatgttgctctactacctttccatgtgttgaaatt

SEQ ID NO: 10 - protein, 1318 aa, Arabidopsis thaliana
  misc_feature: AtELP1
MKNLKLFSEVPQNIQLHSTEEVVQFAAYDIDQSRLFFASSANFVYALQLSSFNESAGAKSAMPVEVCSIDIEPGDFITAFDYLAEKESLLIGTSHGLLLVHNVESDVTELVGNIEGGVKCISPNPTGDLLGLITGLGQLLVMTYDWALMYEKALGEVPEGGYVRETNDLSVNCGGISISWRGDGKYFATMGEVYESGCMSKKIKIWESDSGALQSSSETKEFTQGILEWMPSGAKIAAVYKRKSDDSSPSIAFFERNGLERSSFRIGEPEDATESCENLKWNSASDLLAGVVSCKTYDAIRVWFFSNNHWYLKQEIRYPREAGVTVMWDPTKPLQLICWTLSGQVSVRHFMWVTAVMEDSTAFVIDNSKILVTPLSLSLMPPPMYLFSLSFSSAVRDIAYYSRNSKNCLAVFLSDGNLSFVEFPAPNTWEDLEGKDFSVEISDCKTALGSFVHLLWLDVHSLLCVSAYGSSHNKCLSSGGYDTELHGSYLQEVEVVCHEDHVPDQVTCSGFKASITFQTLLESPVLALAWNPSKRDSAFVEFEGGKVLGYASRSEIMETRSSDDSVCFPSTCPWVRVAQVDASGVHKPLICGLDDMGRLSINGKNLCNNCSSFSFYSELANEVVTHLIILTKQDFLFIVDTKDVLNGDVALGNVFFVIDGRRRDEENMSYVNIWERGAKVIGVLNGDEAAVILQTMRGNLECIYPRKLVLSSITNALAQQRFKDAFNLVRRHRIDFNVIVDLYGWQAFLQSAVAFVEQVNNLNHVTEFVCAMKNEDVTETLYKKFSFSKKGDEVFRVKDSCSNKVSSVLQAIRKALEEHIPESPSRELCILTTLARSDPPAIEESLLRIKSVREMELLNSSDDIRKKSCPSAEEALKHLLWLLDSEAVFEAALGLYDLNLAAIVALNSQRDPKEFLPYLQELEKMPESLMHFKIDIKLQRFDSALRNIVSAGVGYFPDCMNLIKKNPQLFPLGLLLITDPEKKLVVLEAWADHLIDEKRFEDAATTYLCCCKLEKASKAYRECGDWSGVLRVGALMKLGKDEILKLAYELCEEVNALGKPAEAAKIALEYCSDISGGISLLINAREWEEALRVAFLHTADDRISVVKSSALECASGLVSEFKESIEKVGKYLTRYLAVRQRRLLLAAKLKSEERSVVDLDDDTASEASSNLSGMSAYTLGTRRGSAASVSSSNATSRARDLRRQRKSGKIRAGSAGEEMALVDHLKGMRMTDGGKRELKSLLICLVTLGEMESAQKLQQTAENFQVSQVAAVELAHDTVSSESVDEEVYCFERYAQKTRSTARDSDAFSWMLKVFISP

SEQ ID NO: 11 - DNA, 1860 bp, Arabidopsis thaliana
  misc_feature: ELP5
gtgtaaattaagaaccttaaatgtccaaatcatcatcaggatcttcatcagaatcaggatgctcatcgtctgaatctctgaaatatataatctcaccacccttacccgacgaaaccacatcggtctgcaactccattgacgacaatggtgttgtctcaatcttgccatccacaagagatctcctcccatcatagatctcgttcgattttccatcatctgaagaacagagaagtatcaacaacatgttttatccacaatctcaaaaggcttcacagtatctacactcaccttggtgctcaaaaggaagcacaactttttctttctcgacccgttctttctcagacaactggagattgaattgaacctgaaccaaaagaaacaatgatcttatgccatacaacttacgaacaagagatcacattatagcaagagacacagaccaagtaattaaaaagcagatctaggtaacttgccttaggcaaaaggcttttagtggctgcaataacagtatccacagaggaaatgggtgaaaagtttatccccgattgatcaacatgatattcttcagactgtaggtaaagagttacaagagtgttaactaaacgagaatgatagggcatagagagcagagcaagagaaaacttaccattactctcacacgtccttttctaagcttaaaccggacatgaaaccgtcctttaccgaaatcctgatgaaccgaaaagaggttttctaaagcattcctttgcccatctgaagatggacacaaaggttctaagttcgctttcattgtggatatatattcaagcgcatttgtgaccttctcttggtgaaggtcagtgttcaatgaccaaaagacactggaaatttgcgctgcaccgaaaaaaatcaacctttgtagttaaacctaacaaggaataaaactaataaactagtgctcaggtttatgtagcaaagtaccatggcttcgaagatctgttaaaagaccagaaactaatggcatcgctgaatgtcttagcaactcatttacctgattcaaagtttagatatataatcacttctcttttctatagatgatgtctaaacaattagagcttaactcaccgaatcaatggcaacgcagaaacgtgtcttcccagttccaaccaactctgcaccaaagcaatcacttttaatctcatataatcctttagataaagatctaactgaaaaaggattcaatgtaaaagaagtacacctcttcctgcttcaatgattgaagaaaaaagctttttcaagtcactcacacacttatgtaacttaatcaaacttgaaccttcactaaaactagttgaagattgatcaatccatcccaatggatcagtgtaacaatccaaaatacgaatccttcaaatcaaaaccaattgttatcaaacaagtatcacagtgattcgagcaatcaaactactccacaagctaggataatatttagaaaacctaataactcaccatttagaagatgaagagacaacgattcctttttgcttcaacaattgcaaatagaacgaagggctccgcgagaatgtgatcaacacaagacccctgaatcgcaaagtgggaaaaacgaagactttagcagtaatgaagcaattggagatcgaatatagaagaattagggtttaagagtatactgcgacgaggattttccagctaaaattgaagaagatagatttgtgagaagatagccggagacatcgagtccgaaaggggaagctacagtttcttctatagtgagagctggtgcgagttcgccttcttcgccaccatctctgagctttctgaaaatcgattccgccattgttgagagattctagagctctc

SEQ ID NO: 12 - protein, 392 aa, Arabidopsis thaliana
  MISC_FEATURE: ELP5
MAESIFRKLRDGGEEGELAPALTIEETVASPFGLDVSGYLLTNLSSSILAGKSSSQGLVLITFSRSPSFYLQLLKQKGIVVSSSSKWIRILDCYTDPLGWIDQSSTSFSEGSSLIKLHKCVSDLKKLFSSIIEAGRVRSLSKGLYEIKSDCFGAELVGTGKTRFCVAIDSKREVIIYLNFESAQISSVFWSLNTDLHQEKVTNALEYISTMKANLEPLCPSSDGQRNALENLFSVHQDFGKGRFHVRFKLRKGRVRVMSEEYHVDQSGINFSPISSVDTVIAATKSLLPKASYLDLLFNYLVQFNLQLSEKERVEKEKVVLPFEHQDDGKSNEIYDGRRSLVDGKIETTPLSSMELQTDVVSSGKGGEIIYFRDSDDEHPDSDEDPDDDLDI

SEQ ID NO: 13 - DNA, 1009 bp, Arabidopsis thaliana
  CDS (13)..(801)
ccaaatttctccatggatcgttctttgaatctcctcgatttagccttagggttcgatgagcagctagctattccatcgccactaaatggaaaagtaatactaatagaagactgtgtagagacgagtggttcctttgtacttcaccagctaatgaaacgtgttctctcctctaactcctccgacgcacttatctttctcgcttttgctcgccctttctctcattatgatcgaatcttgcgtaaactgggatgtaatttagctacccataagtcgaataatcgattggtgttctttgacatgctcatggttaagtgttcagatggggatcaaatggaagacaatgtgagtgcagttgcgaaactatttcgggagatacaagaaaccgttcgaaagctacagagtgtaacaagtggtaacataactgttatggtggatgacatgtctctgctggaaattgctactaccggcagcaactcagatcacgtattggacttcttgcattattgccacacattaagttctgaaagcaattgttcattggtcatcctcaatcatgaagatatatacgcgagcatggagagacctgcatttttgctacagatggtatgccttgcagatgttgtgataaaggcagagcctttagcctctggtttagcaaatgatgtacatggccaattgactgttctgaacaaagggataagcaactcaggtagaggaagctcgaggaacaagttgcagaattttcaattcaggatcaaagaaaatggtatcgactatttctatcctggttgcagaagctgaggattagacaccgttctgagcattagcatggtagtgagatgtttttctagacagttttgttatttgcgtttaggttaaacagagacactctgttaaacatttttactttagtgtctcttctggttgtaacttgatgacttagctaaagttgaatgcttgagacacagaaacttgtagacatggaaaattagtaacattctatatcttt

SEQ ID NO: 14 - protein, 262 aa, Arabidopsis thaliana
  misc_feature: ELP6
MDRSLNLLDLALGFDEQLAIPSPLNGKVILIEDCVETSGSFVLHQLMKRVLSSNSSDALIFLAFARPFSHYDRILRKLGCNLATHKSNNRLVFFDMLMVKCSDGDQMEDNVSAVAKLFREIQETVRKLQSVTSGNITVMVDDMSLLEIATTGSNSDHVLDFLHYCHTLSSESNCSLVILNHEDIYASMERPAFLLQMVCLADVVIKAEPLASGLANDVHGQLTVLNKGISNSGRGSSRNKLQNFQFRIKENGIDYFYPGCRS

SEQ ID NO: 15 - DNA, 1302 bp, Arabidopsis thaliana
  promoter (1)..(1302): DRL1 promoter
gagctcttgaacatcttcagcgtcaaacgttcgcttgaaacccattcttaaaaaacctgtaaaaggagaaccaaagccattagttacttggcgtgaaggttatctgtgtgaaccacactcacactgacaagtgacaactataagtctatttaaccagttcagtaattaaattagagtataaagaaaaaatatttaaccccaattaatttaagtaaattaaaaaaaaaaatccccaaatttgaaaaccctaaccctagatttcaattcacggattaaatcgcgaaatcaagaatcgaaaatcaatgaatcggttaattatcatgaatacagacaaaagccctcgattaattgacagatctgacgaaccctagaacatcccaacctacgagcccagatcggataataaaattgggggaaactgaaattaagaacggatccgttcatacttttgaaggaaatcaacatcaaaaacaaacaattgatcagatctgttgttatcagatccagatctgactcaaaccctagatttgaccaaaacaaagggatgataacaaaacaggtatcaattggaagatcaaatccagaggaagaagaaaattacaatagatctgaagatcgatctggaagaaggaagggatctgagctgaaattcgaagaaaatttggtaggggaaaaaaataaaattgggggaattttcgaaagcggctacggttgaaaattcgtgcttttcgattaggggaaagacccgattttgaaacccggatttaactcggataccgagacaacatcgaatgagctgtactttatttaaattctttgaattacttgaatttaccaaattatcctcaccgtataccatcaacgtgcctagcaacttgtgttggcatatccggctcgtatctatcttctaaaccggacacaaattgatattcatatgaacaaaccggaccaagcgaattaaaccggttctaatctataagccctagattcccaaatcaaattttcacttttgctttcttcaacctgtcgacaacactcccgaatttttctctggtaatgtttttattaacttgatttttcgtcggaattgataattgtggaagtatgtactaataaagtttgatactttaacttgttctggggattgaatccgttatgatatgaatttctggaattttagtttttatatgggaatctaaagcttattcgtttgttatgtgttgatgttcttatgcacctaaagcttgaattgaaatagtcttgtattgattccatttttgtaggcagtgtgtttaaaaacca

SEQ ID NO: 16 - protein, 39 aa, Arabidopsis thaliana
  MISC_FEATURE: conserved sequence of DRL
EKNLRGKLRSDVDRSVSTGEIVIVDSLNSIKGYRYELWC

SEQ ID NO: 17 - DNA, 23 bp, Artificial Sequence
  primer8
gtgggcaaccttgtagtggtaag

SEQ ID NO: 18 - DNA, 26 bp, Artificial Sequence
  primer2
ttcgtttccgtcccgcaagttaaata

SEQ ID NO: 19 - DNA, 20 bp, Artificial Sequence
  primer3
ttttgtaggcagtgtgttta

SEQ ID NO: 20 - DNA, 20 bp, Artificial Sequence
  primer4
tcgtcgttttatgattttat

SEQ ID NO: 21 - DNA, 23 bp, Artificial Sequence
  primer5
agccccaaaatatgtttgcatta

SEQ ID NO: 22 - DNA, 23 bp, Artificial Sequence
  primer6
tcgcgttgatgatttcttgtgtc

SEQ ID NO: 23 - DNA, 23 bp, Artificial Sequence
  primer7
ctagaccgcaaccaaaactatgc

SEQ ID NO: 24 - DNA, 30 bp, Artificial Sequence
  primer1
cgggattttcccatcctactttcatccctg

SEQ ID NO: 25 - DNA, 23 bp, Artificial Sequence
  primer9
actagcgccatgggtttttaaac

SEQ ID NO: 26 - DNA, 26 bp, Artificial Sequence
  primer10
tagttacttggcatgcaggttatctg

SEQ ID NO: 27 - DNA, 22 bp, Artificial Sequence
  primer11
aaggagaaccaaagccattagt

SEQ ID NO: 28 - DNA, 22 bp, Artificial Sequence
  primer p12
gcattagcgattaatgaagctg

SEQ ID NO: 29 - DNA, 21 bp, Artificial Sequence
  primer used for pGAD10 insert amplification
gcacagttgaagtgaacttgc

SEQ ID NO: 30 - DNA, 26 bp, Artificial Sequence
  primer used for DRL1 gene coding sequence amplification
atttgtatgattaaaagtaagctgca

SEQ ID NO: 31 - DNA, 21 bp, Artificial Sequence
  primer used for pGAD10 insert amplification
agggatgtttaataccactac

SEQ ID NO: 32 - DNA, 26 bp, Artificial Sequence
  primer used for DRL1 gene coding sequence amplification
gtttaaaaacccatggcgctagttgt

SEQ ID NO: 33 - protein, 313 aa, Saccharomyces cerevisiae
MPLVLFTGYPCSGKTTLAKHLVQLLQSKIDATPSLSKYSITYHSDESLGIKHSDYITSQDERKLRSEIISAVKRDLSKNKIVIVDSLNYIKGFRYQLHCEVKNLSTTFCVIQTLCPPETIFEWNKTSNPNPWEPELLNQLIQRYEEPNSSNRWDSPLFAILTPQDNITDYIDDICKVVFQTSKSAKNSGHNDPLSKGLQKPNSATVLKPASQSNFIQVLDIETSKIIKTIMNHIKSLTSIGGVSNGTRVIVSEGITDINDDGCFFVDLPIGNVVTLAQLQRLKRQFINFNKLRDIDQDRIGPLFADYLNKNLN

SEQ ID NO: 34 - protein, 281 aa, Schizosaccharomyces pombe
MPLIIVSGYPSSGKTTRSNELKKALEDRIHQNIDNTKDYRVIIINDESLNIEKETYRESKNEKAARGLLYSAVQRELSKSTFVICDALNYIKGFRYQLYCESKSMYTTHCVIHVAVPQDLCRKFNSNKEQPYPDDVLEQLMFRYEEPNGMTRWDSPLFTVLHDDASCPIDDIWSVLIHNKNVKPNQATMVKPPAEVNYLYELDKTTQDVIMLILDNSNDTSLITVPGSKLQIALPSVTVSLPLLQRLRRQFIQINRQQSYNTNVLKEMFVEFLNGQFETLD

SEQ ID NO: 35 - protein, 299 aa, Drosophila melanogaster
MPLVVITGLPASGKSTRARQLRDHFVERGRKVHLISENEAVPKAGFGKNSHTGDSQKEKVVRSDLKSEASRHLNQEDLVILDAGNYIKGYRYELYCMSKVSRTTQCTVFTCIPQEEAWTFNSQRTAPDELPGDSERVQPVDNSDVPYTRETFDALCQRYEEPQSNNRWDSPLVVVLPKDTLDMEAIYKALYESQPLPPNQSTYNAPLGTTNYLFELDKIVQAIIKEILGAVKIKAFGQLRIPGSRNPVKVATSMNALQLNRLRQKFITSTCHASQTSPTPLEQVPHLFVQFINANTIGC

SEQ ID NO: 36 - protein, 351 aa, Mus musculus
MPLVVVCGLPSSGKSQRTEELRRALASEGRAVYVVDDASVLGAQDPTVYGDSAGEKALRAALRAAVERRLSRQDVVILDSVNYIKGFRYELYCLARAARTPLCLLYCVRPNWPSRELREASASENRDLAVSVSWRPRAETGGKSQAAGAVEEQRATSPVANGGVLAAVSKELDPEEILPSNPSAVMTPGSEKSAEPASCAFPPEVLESLALRFESPDSRNRWDRPLFTVVGLEEPLPLAEIRSALFENRAPPPHQSTQSQPLASGSFLHQLDQATSQVLTAVMEAQKSAVPGDFLKLPGTTEPLRFTRPLTLAELSRLRRQFISYTKMHPNNENLPQLANMFLQYLNQSLH

SEQ ID NO: 37 - protein, 354 aa, Homo sapiens
MPLVVFCGLPYSGKSRRAEELRVALAAEGRAVYVVDDAAVLGAEDPAVYGDSAREKALRGALRASVERRLSRHDVVILDSLNYIKGFRYELYCLARAARTPLCLVYCVRPGGPIAGPQVAGANENPGRNVSVSWRPRAEEDGRAQAAGSSVLRELHTADSVVNGSAQADVPKELEREESGAAESPALVTPDSEKSAKHGSGAFYSPELLEALTLRFEAPDSRNRWDRPLFTLVGLEEPLPLAGIRSALFENRAPPPHQSTQSQPLASGSFLHQLDQVTSQVLAGLMEAQKSAVPGDLLTLPGTTEHLRFTRPLTMAELSRLRRQFISYTKMHPNNENLPQLANMFLQYLSQSLH

SEQ ID NO: 38 - DNA, 21 bp, Artificial Sequence
  primer used for actin cDNA amplification
gtgccaatctacgcgggtttc

SEQ ID NO: 39 - DNA, 21 bp, Artificial Sequence
  primer used for actin cDNA amplification
caatgggactaaaacgcaaaa

SEQ ID NO: 40 - DNA, 23 bp, Artificial Sequence
  primer used for amplification of the DRL1 gene
tcgcgttgatgatttcttgtgtc

SEQ ID NO: 41 - DNA, 23 bp, Artificial Sequence
  primer used for amplification of the DRL1 gene
ctagaccgcaaccaaaactatgc

SEQ ID NO: 42 - DNA, 21 bp, Artificial Sequence
  primer used for amplification of the AN gene
tgagacggtgccgtggtatgg

SEQ ID NO: 43 - DNA, 20 bp, Artificial Sequence
  primer used for amplification of the AN gene
gttgcctactggtggattcc